(19) United States
(12) Patent Application Publication
(10) Pub. No.: US 2022/0111093 A1
(43) Pub. Date: Apr. 14, 2022

(54) STERILIZATION PROCESS, SYSTEM AND PRODUCT INCLUDING MOLECULAR MOBILITY ENHANCER

(71) Applicant: TekDry International, Inc., Lafayette, CO (US)

(72) Inventors: Daniel STOREY, Longmont, CO (US); Christina K. THOMAS, Iron River, WI (US)

(21) Appl. No.: 17/423,803

(22) PCT Filed: Jan. 28, 2020
(86) PCT No.: PCT/US2020/015462

§ 371 Date: Jul. 16, 2021

Related U.S. Application Data
(60) Provisional application No. 62/797,789, filed on Jan. 28, 2019.

(51) Int. Cl. A61L 2/20; A01N 25/08; A01N 37/16

(52) U.S. Cl. CPC A61L 2/208; A01N 25/08; A01N 37/16; A61L 2202/18; A61L 2202/26; A61L 2202/122; A61L 2202/24

(57) ABSTRACT

Methods for generating a sterilant vapor in a sub-atmospheric environment for use in sanitization and sterilization. The vapor is generated from a mixture containing a sterilant and a molecular mobility enhancer (MME). More specifically, the use of the MME enhances the vaporization and mobility of the sterilant, particularly hydrogen peroxide and/or a peroxy acid, to provide enhanced or improved sterilization. Also provided are solid-form sterilant comprising a sterilant and an MME. Further provides are various packaged forms of sterilant, particularly containing hydrogen peroxide and/or a peroxy acid for use in sanitation and sterilization methods herein.

### BACKGROUND

Described in this disclosure are processes for generating a sterilant vapor from a peri-peroxyacid liquid solution which includes a peroxy acid and a molecular mobility enhancer (MME) for sterilization of various items under vacuum including, health care and/or medical equipment and devices. It particularly relates to a process by which the use of an MME enhances the vaporization and mobility of hydrogen peroxide or a peroxy acid, such as performic acid, under reduced pressure and low temperatures to provide enhanced or improved sterilization.

The proper sterilization of medical devices, surgical instruments, supplies and equipment utilized in direct patient care and surgery is a critical aspect of the modern health care delivery system and directly impacts patient safety.

The Association for the Advancement of Medical Instrumentation (AAMI) defines sterilization as: “A process designed to remove or destroy all viable forms of microbial life, including bacterial spores, to achieve an acceptable sterility assurance level.” Sterility is measured by probability expressed as sterility assurance level (SAL). It is generally accepted that a sterility assurance level (SAL) of 10⁻⁶ is appropriate for items intended to come into contact with compromised tissue, which has lost the integrity of natural body barriers. This would include sterile body cavities, tissues and vascular system. A sterility assurance level (SAL) of 10⁻⁶ means that there is less than or equal to one chance in a million that a particular item is contaminated or unsterile following a sterilization process.

Contexts in which medical devices are reused, including where surgical instruments enter normally sterile tissue or the vascular system (e.g., endoscopes, catheters, etc.), typically require sterilization before each use. Improperly sterilized or contaminated medical devices utilized in patient care can contribute to surgical site infection and can pose a serious risk to the patient's safety and welfare, which can result in a serious life-threatening infection or even death. Proper sterilization of items having diffusion restricted areas, such as lumens in medical devices and electronic components, such as 3D protoboards, can represent a significant challenge.

Some sterilization processes can be complex and/or involved. The effectiveness of such processes can often involve thorough training of healthcare workers and technicians involved in the reprocessing and sterilization of medical devices, including providing them with continually updated knowledge and understanding of the scientific principles and methods of sterilization utilized in today's health care settings. For example, use of many sterilants and sterilizing equipment can involve health hazards and other inherent risks, which can be greatly minimized through proper education, precautions, policies, etc. Sterilization processes can typically be used wherever patient care is provided and/or wherever infection control is otherwise desired, such as in acute care hospitals, ambulatory surgical centers, outpatient facilities, dental or physician's offices, etc.

Effective sterilization processes, particularly in medical settings, can rely on various conditions. One such condition is ensuring that the sterilization environment is suited to effectively destroy living organisms. For example, some processes rely on the sterilant and sterilizing equipment being validated and appropriate in design and operation to achieve the correct combination of temperature and sterilant combination (and/or other environmental conditions) to be lethal to microorganisms. Another such condition involves thoroughly cleaning the devices to be sterilized to reduce bioburden (e.g., soil). Larger bioburden can frustrate the sterilization process. If bioburden is too great, the established sterilization parameters may not be adequate for effective sterilization. Another such condition involves providing and maintaining intimate and adequate contact between the sterilant and all surfaces and crevices of the device to be sterilized. In practice, different sterilization processes can be used in different contexts (e.g., to yield a different desired SAL), and different processes can require or desire certain conditions.

There remains a need in the art for effective methods for sterilization of a variety of items, including medical devices, such as endoscopes, and electronic equipment.

### SUMMARY

It has been recognized by the inventors that exceptional sterilization of a wide range of materials can be achieved using peri-peroxy acid vapor in a vacuum based sterilization system. The sterilization system converts a peri-peroxy acid solution to a vapor using an outgassing process that deliberately and in a controlled manner releases sterilant vapor from a solution or a solid. It has further been recognized by the inventors that the mobility and transport of the peroxy acid vapor in a vacuum environment is enhanced by combining the peroxy acid with a Molecular Mobility Enhancing (MME) agent. The vaporous MME agent assists in the transport of the peroxy acid vapor throughout a vacuum chamber and further assist in the infiltration of the peroxy acid vapor into intricate devices to provide enhanced sterilization. In an embodiment, enhanced sterilization is a desired level of sterilization, for example as measured by achieving a desired SAL, in a shorter time than sterilization not employing such an MME agent.

Among other things this disclosure addresses certain shortcomings of peroxy acids for sterilization, especially performic acid, such as the stability of this liquid for use in a vapor sterilization system. It is known that performic acid liquid is difficult to employ as a sterilant solution due to its highly reactive and unstable nature. Performic acid, which is not commercially available, is unstable and decomposes nearly immediately upon standing and must be used within a limited time after synthesis. As a result, solutions of formic acid and hydrogen peroxide must be mixed just prior to use (in situ). The highly reactive nature of performic acid when heated, the limited amount of time that one has to utilize the sterilization solution once synthesized, and the hazards of handling unstable liquid solutions are several drawbacks to using performic acid in current methods that use sterilant vapors. In the present disclosure, methods for overcoming limitations of using performic acid as the peroxy acid component of choice as part of the peri-peroxy acid sterilant solution are described. Enhancing stability of the peroxy acid component for use independently or as part of the peri-peroxy acid solution may be overcome by using a stabilizer and/or encapsulating the peroxy acid independently or a peri-peroxy acid solution (e.g., including other sterilants and/or an MME agent) in a stabilization matrix for use in a reduced pressure sterilization system if a longer term storage requirement is needed. The present disclosure also describes methods for packaging and delivering the component chemicals for “on demand” generation of the peri-peroxy acid sterilant solution for use inside of the sterilization system.

Performic acid is an effective sterilizing agent component for the composition of the peri-peroxy acid vapor source. Other effective organic peroxy acid(s) can be vaporized from a mixture containing peroxy acid, including but not limited to, any number of other saturated and unsaturated peroxy acids having 1 to 8 carbon atoms (C1-C8 peroxy acids) and including any halogenated forms of the peroxy acids, so long as the molecule(s) being utilized are adequately volatilized to form a vapor of a concentration necessary to sterilize the device in question under reduced pressures in a vacuum sterilization system as described herein, with or without the assistance of heat at a temperature of equal to or less than what is required by the device being sterilized. Some examples of other peroxy acids include, but are not limited to, peroxyacetic acid and its halogenated derivatives, peroxypropionic acid, and its halogenated derivatives and peroxybutyric acid and its halogenated derivatives. Halogenated peroxy acids may contain one or more fluoro-, chloro-, bromo- and/or iodo-groups. Other sterilizing agents that may also be vaporized as part of a mixture may include, but are not limited to, the peroxy acid parent carboxylic acid, hydrogen peroxide, and alcohols.

Additionally, this disclosure relates to improved sterilants containing H₂O₂ (hydrogen peroxide) and an MME and methods of sterilization under reduced pressure using such improved sterilants.

It has been found that the use of MME allows for faster sterilization time with a lower amount of sterilant. This is particularly, the case when the sterilant is hydrogen peroxide and the MME is methanol.

The disclosure further relates to methods for providing a sterilant vapor to an enclosure, the method comprising exposing a mixture comprising a molecular mobility enhancer (MME) and the sterilant to the enclosure at a sub-atmospheric pressure condition, thereby providing the sterilant as a vapor in the enclosure. In an embodiment, the exposing step provides the sterilant and the MME as the vapor in the enclosure. In an embodiment, the exposing step results in transport of the sterilant within the enclosure. In an embodiment, the exposing step results in contact of the sterilant with surfaces of the enclosure and/or with surfaces of an article within the enclosure. In an embodiment, the exposing step sterilizes or sanitizes the enclosure and/or an article provided within the enclosure. In embodiments, the method further comprising heating the mixture during the exposing step. In embodiments, the method further comprising providing a carrier gas flow in fluid communication with the mixture and flowing the carrier gas flow in fluid communication with the mixture into the enclosure. In embodiments, the method further comprising maintaining the enclosure at a pressure selected over the range of 0.1 torr to 200 torr for a time period selected from the range of 1 minute to 24 hours. In embodiments, in the method the exposing step comprises providing the mixture in fluid communication with the sub-atmospheric enclosure, thereby providing for transport of the sterilant into the enclosure. In embodiments of the method, the exposing step comprises providing the mixture in the enclosure followed by decreasing the pressure of the enclosure to below 760 torr. In embodiments, the pressure of the enclosure is decreased to a pressure selected over the range of 0.1 torr to 200 torr. In embodiments, the enclosure is a vacuum chamber or a processing chamber. In embodiments, the article provided within the enclosure is a medical device or component thereof. In embodiments, the medical device is an endoscope or component thereof.

In embodiments of method herein, the mixture of sterilant and MME used to provide the sterilant vapor in the enclosure is a solid-form sterilant in any embodiment described herein. In embodiments of method herein, the mixture of sterilant and MME used to provide the sterilant vapor in the enclosure is a packaged sterilant comprising sterilant and MME as described in any embodiment herein.

In embodiments, the disclosure provides a method for delivery of a sterilant into an apparatus (enclosure, chamber or vacuum chamber) capable of achieving reduced pressure which comprises introducing a mixture of a sterilant and a molecular mobility enhancer into the apparatus and reducing the pressure in at least a portion of the apparatus to generate a vapor comprising sterilant in at least a portion of the apparatus. In embodiments, the vapor generated comprises sterilant and MME. In embodiments, an article to be treated is present in the apparatus and the vapor generated is in contact with the article. In embodiments, the article to be treated comprises diffusion resistant surfaces. In embodiments, the mixture of sterilant and MME is any solid-form sterilant as described in any embodiment herein. In embodiments, the mixture of sterilant and MME is provided to the apparatus as a packaged sterilant as described in any embodiment herein.

In embodiments, the disclosure provides a method of sterilizing an article by contacting the article with a vapor comprising a sterilant and an MME under vacuum at a selected pressure of the vapor for a selected time. In embodiments, the vapor is generated under vacuum from a mixture of sterilant and MME. In embodiments, the mixture is a solid-form sterilant of any embodiment described herein. In embodiments, the mixture of sterilant and MME is provided to the apparatus as a packaged sterilant as described in any embodiment herein.

In embodiments, the sterilant is a liquid at normal temperature and pressure (NPT, 20° C. and 760 torr). In embodiments, the sterilant is a solid at normal temperature and pressure (NPT, 20° C. and 760 torr). In embodiments, the MME is a liquid at normal temperature and pressure (NPT, 20° C. and 760 torr). In embodiments, the MME is a solid at normal temperature and pressure (NPT, 20° C. and 760 torr).

In embodiments, the molecular mobility enhancer is one or more of an alcohol, alkane, carboxylic acid, ester, ether, ketone and any combination thereof.

In embodiments, the molecular mobility enhancer is one or more of: a C1-C20 alcohol, a C5-C20 alkane, a C1-C20 carboxylic acid, a C3-C20 ester, a C4-C20 ether, a C3-C20 ketone and any combination thereof.

In embodiments, the molecular mobility enhancer has a vapor pressure equal to or greater than 10 torr at 20° C. and 760 torr. In embodiments, the molecular mobility enhancer has a vapor pressure equal to or greater than 100 torr at 20° C. and 760 torr. In embodiments, the sterilant has a vapor pressure equal to or greater than 10 torr at 20° C. and 760 torr. In embodiments, the sterilant has a vapor pressure equal to or greater than 100 torr at 20° C. and 760 torr.

In embodiments, the sterilant comprises hydrogen peroxide, a peroxy acid, a halogenated peroxy acid, a carboxylic acid, or an alcohol or any combination thereof. In embodiments, the sterilant is selected from the group consisting of a peroxy acid, a phenolic acid, hypochlorous acid, isopropanol, hydrogen peroxide, glutaraldehyde, ortho-phthaladehyde, and combinations thereof. In embodiments, the sterilant comprises hydrogen peroxide or is hydrogen peroxide. In embodiments, the sterilant comprises a peroxy acid or is a peroxy acid. In embodiments, the sterilant comprises performic acid or is performic acid. In embodiments, the sterilant comprises peracetic acid or is peracetic acid. In embodiments, the sterilant is one or more of a peroxide, peroxyacid, alcohol, chlorine-containing compound, a phenolic compound and any combination thereof. In embodiments, the molecular mobility enhancer is methanol, diethyl ether or methylmethanoate or a combination thereof and the sterilant is hydrogen peroxide, a peroxy acid or a mixture thereof.

According to one set of embodiments, a sterilizing system is provided for sterilizing medical instruments. The system includes: a chamber configured to receive a medical instrument; a pressurization subsystem configured, when the medical instrument is in the chamber, to produce a negative pressure environment within the chamber sufficient to gasify liquid and contamination on and in the medical instrument; and a heating subsystem configured to generate heat and comprising a thermal conduction assembly configured, when the medical instrument is in the chamber to conduct heat to the medical device. In one arrangement, the thermal conductions assembly is further configured to at least partially conform to an external shape of the medical device. In addition, a sterilizing subassembly is provided that off-gasses sterilant in a negative pressure environment. In an embodiment, the sterilizing subassembly may include a package of solid-form sterilant within a matrix consisting of, for example, a polymer or other material. The sterilizing subassembly may include a package of liquid sterilant (e.g., which may be encapsulated within a matrix) that may be dispersed into the negative pressure environment. In any embodiment, the sterilant is in some form a liquid with a sufficiently high vapor pressure such that the outgassing, assuming adiabatic expansion of the gas, reaches all surfaces of the product, thus providing a sterile environment. In a further arrangement, the sterilant may further include a molecular mobility enhancing agent the facilitates the evaporation and/or transportation of the sterilant. In one arrangement, the sterilizing subassembly further includes a heater (e.g., conductive heater or radiant heater) for heating the solid form-sterilant. In such an arrangement, the medical instrument and the solid-form sterilant may be heated to different temperatures. In another arrangement, the sterilizing subassembly includes a separate chamber that may be pressurized to a negative pressure. In this arrangement, the two chambers may be selectively connected and/or isolated.

### BRIEF DESCRIPTION OF THE DRAWINGS

FIG. 1 shows a block diagram of the sterilization/sanitization process 100 in the Sterilizing System 105. This system includes a number of subsystems that create the entire system. It is understood this is a representation of one embodiment and it is not necessary to have all the subsystems in place for the entire system to function in other methods. A Heating Subsystem 140, Monitoring Subsystem 160, Pressurizing Subsystem 130, User Interaction Subsystem 150, Sterilizing Subassembly 170 and an MME Subassembly 180 are all connected physically and/or electronically to the Sterilizing Chamber 110 through normal means. The Device 120 is placed into the Sterilizing Chamber by the User 103 and the sterilizing/sanitizing process is started by the User 103 through the Communication Subsystem 190. The process is controlled through a set of pre-programmed routines via the Controller 180 which is typically a programmable logic controller (PLC) that can be purchased off the shelf.

FIG. 2 illustrates a non-limiting exemplary medical device is an endoscope 120. An endoscope is an example of an item having diffusion restricted areas.

FIGS. 3A and 3B shows diagrams of exemplary, but not limiting, sterilizers and how the subsystems connect to the process chamber 110 and 110a, respectively.

FIG. 4 shows a block diagram flow chart 500 of the generic process for sterilization of a device using the MME. The same process can be used for sanitization, with an adjustment of the specific process parameters for sanitization versus sterilization.

### DETAILED DESCRIPTION

Embodiments disclosed provide systems, methods and sterilants for sterilizing medical devices (e.g., electronic or non-electronic medical device) or other instruments within a negatively pressurized chamber that includes an evaporable sterilant. The disclosure sets forth a description of one exemplary sterilization system that may utilize the disclosed sterilants. The disclosure further discusses processes for generating a sterilant vapor from a peri-peroxyacid liquid solution whose contents include a peroxy acid and a molecular mobility enhancer (MME).

As discussed herein, the pressurized chamber can apply negative pressure to cause any liquid on or in the device to gasify and leave the device, destroying any bacteria, while a conductive heating assembly supplies heat to the device. In some implementations, heat supplied by the conductive heating assembly can be enough to avoid freezing during removal of liquid from the device. In other implementations, additional heat is applied to the device to further aid the sterilization. Some embodiments of the conductive heating assembly are designed to gently and evenly supply conductive heat to the device without damaging the device, for example, through scratching, overheating, etc. Additionally, the evaporable sterilant used can be delivered locally, thus avoiding the issue of corrosion or other issues these sterilants can cause in higher concentrations.

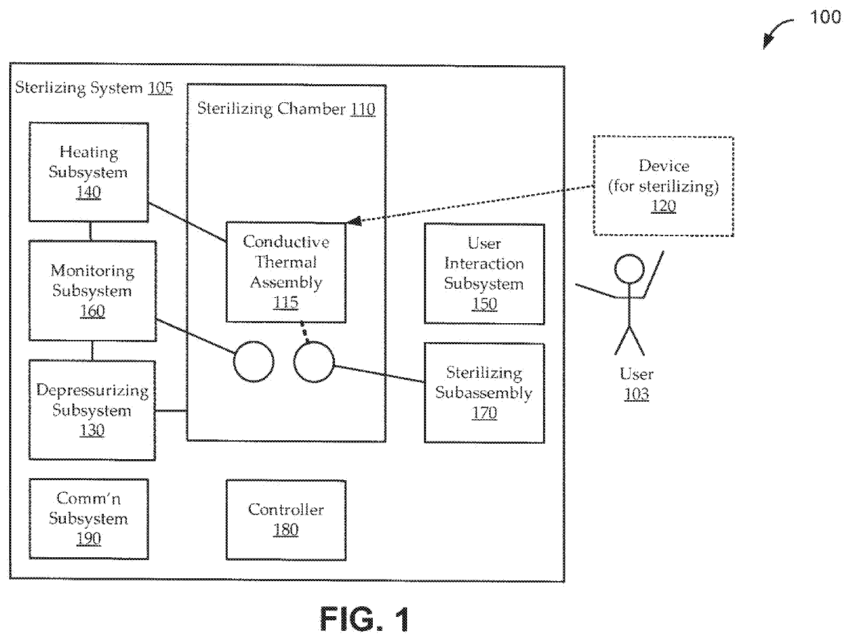

Turning first to FIG. 1, a block diagram is shown of an embodiment of a sterilizing environment 100, according to various embodiments. The sterilizing environment 100 includes a sterilizing system 105 that can be used by users 103 to dry and/or sterilize any one or more suitable device(s) 120. The device(s) 120 can be medical devices including electronic components, non-electronic medical devices, instruments, and other medical or non-medical items. For example, the sterilizing system 105 can be used to sterilize devices 120 that have been overexposed to microbial contaminants (e.g., including liquid contamination), cleaning solutions, etc. The device(s) 120 can be placed into a sterilizing chamber 110 where contact is established with a conductive thermal assembly 115 and sterilizing subassembly 170. As described herein, in some implementations, the sterilizing subassembly 170 is a separate assembly from the sterilizing chamber. In other implementations, the sterilizing assembly 170 is positioned within the sterilizing chamber. In yet further implementations, the sterilizing assembly is part of the conductive thermal assembly 115 (e.g., as a sterilant coating on thermally conductive beads/conformable media). Negative pressure (e.g., a partial vacuum) is applied to the sterilizing chamber 110 by a depressurizing subsystem 130, and heat is applied to the device(s) 120 via the conductive thermal assembly 115 using a heating subsystem 140. In further implementations, the heating subsystem 140 or a separate heating subsystem may apply heat to the sterilizing subassembly.

The sterilizing subassembly 170 can include any suitable sterilant and delivery mechanism. For example, implementations of the sterilizing subassembly 170 can include sterilant packages (e.g., solid-form sterilants, ampoules, cartridges, etc.) disposed within the chamber, sterilant packages disposed within a separate chamber in fluid communication with the chamber, coated beads, zeolites, ultraviolet radiation sources, vibration, and/or other elements that can be activated in response to low pressure conditions and with sufficient intensity (e.g., concentration, amount, etc.) and/or amount of time to ensure sterilization to at least a predetermined sterilization level (e.g., a SAL of 10-6). In some implementations, the sterilizing subassembly 170 can include a liquid sterilant encased in a polymer or other matrix that can be released (e.g., off-gassed) upon heating, low pressure, or a combination of the two. Such a liquid sterilant disposed within a matrix is sometimes referred to, herein, as a solid-form sterilant. In other implementations, the sterilizing subassembly 170 can include liquid sterilant housed in an ampoule or cartridge

In some embodiments, a monitoring system 160 tracks the sterilization process. For example, the monitoring system 160 can record and log a set of parameters for use in monitoring compliance with a quality process or standard, such as the ISO 13485 quality standards. In some implementations, the set of parameters and/or other information can be fed back (e.g., to the controller 180) to adjust the environment of the sterilization chamber 110.

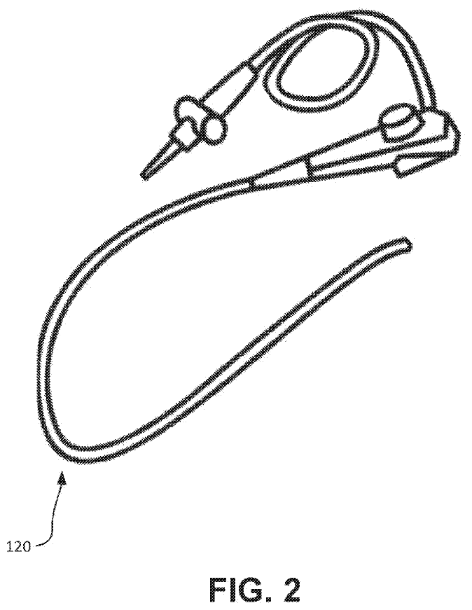

The sterilizing environment 100 can be used to treat any suitable type of device 120 to be sterilized. For example, in a medical context, the device 120 can be an electromechanical device (e.g., insulin pump, injector, etc.), portable computer monitoring system (e.g., tablet, laptop, etc.), surgical implement (e.g., scalpel, trocar, etc.), cauterizer, portable audio and/or video recording device (e.g., voice recorder, camera, video recorder, etc.), portable imaging device, implantable device (e.g., orthopedic implant, etc.), etc. As shown in FIG. 2, one non-limiting exemplary medical device is an endoscope 120. As will be appreciated, many such endoscopic device include various electronic components (e.g., electrodes, cameras etc.), which make sterilizing these devices problematic. Further, the illustrated medical device includes various internal lumens, which have previously frustrated sterilization efforts. Typically, the device 120 has exposure limits (e.g., set by the manufacturer) for one or more environmental conditions, such as temperature, exposure to caustic sterilants, radiation or heat. For example, many devices 120 can have relatively low exposure limits for temperature, caustic environments, liquids, radiation, etc. Accordingly, embodiments can use negative pressure (e.g., vacuum) to facilitate a “cool” flash boiling of liquid inside the device 120; and a controlled, relatively low temperature can be used to facilitate the sterilizing, while remaining well within the thermal exposure limits of the device. At the same time, due to the drop-in pressure from the negative pressure/vacuum, it is possible to release encapsulated sterilant (e.g., liquid sterilant disposed within a matrix, cartridge or ampoule) locally, thus providing sufficient sterilant to sterilize to a desired level without overexposing the device 120 to sterilant, which can cause damage.

Embodiments of the sterilizing chamber 110 are manufactured in any suitable manner in any suitable size and of any suitable shape and material, so that desired number and/or types of devices 120 can fit within the chamber, and the chamber can support the types of negative pressure applied to it by the pressurizing subsystem 130. For example, the sterilizing chamber 110 can be made of metal or sturdy plastic and can include seals, where appropriate, to maintain appropriate levels of negative pressure within the sterilizing chamber 110. Some implementations include multiple sterilizing chambers 110 for concurrent, but segregated sterilizing of multiple devices 120, or for sterilizing of different sizes and/or shapes of devices 12 (e.g., with correspondingly sized and/or shaped sterilizing chambers 110). Some are designed to facilitate use within context of a larger assembly (e.g., a wall-mounted or case-integrated sterilizing chamber 110). In one implementation, multiple sterilizing chambers 110 are stacked in a configuration that allows access like a drawer, chest, etc.). Some implementations further include windows, internal lighting (such as UV light), and/or other features to allow users 103 to view the inside environment (e.g., during sterilizing of their device(s) 120).

The sterilizing chamber 110 is pressurized by a depressurizing subsystem 130. Embodiments of the depressurizing subsystem 130 include a vacuum pump or the like for producing a negative pressure environment within the sterilizing chamber 110. The specifications of the depressurizing subsystem 130 are selected to produce a desired vacuum level within a desired amount of time, given the air-space within the sterilizing chamber 110, the quality of the sterilizing chamber 110 seals, etc. In one embodiment, the depressurizing subsystem 130 includes a one-half-horsepower, two-stage vacuum pump configured to produce a vacuum level within the sterilizing chamber 110 of approximately 0.4 inches of mercury (“inHg”) within seconds and to maintain substantially that level of pressure throughout the sterilizing routine (e.g., for fifteen to thirty minutes). Different depressurizing subsystem 130 specifications can be used to support concurrent sterilizing in multiple sterilizing chambers 110, sterilizing in sterilizing chamber 110 of different sizes, use in portable versus hard-mounted implementations, etc.

In some embodiments, the depressurizing subsystem 130 is in fluid communication with the sterilizing chamber 110 (or multiple sterilizing chambers 110) via one or more fluid paths. For example, a fluid path can include one or more release valves, hoses, fittings, seals, etc. The fluid path components are selected to operate within the produced level of negative pressure. Certain embodiments include an electronically controlled (or manual in some implementations) release valve for releasing the negative pressure environment to allow the sterilizing chamber 110 to be opened after the sterilizing routine has completed (or at any other desirable time). This release valve has the ability to contain a filter such that room air can be allowed in the chamber without causing recontamination.

In another embodiment, the release valve is attached to a container of sterilized, pressurized gas such as pure argon or pure nitrogen. In implementations including multiple sterilizing chambers 110, multiple fluid paths, multiple release valves, or other techniques can be used to fluidly couple the pressurizing subsystem 130 with the sterilizing chambers 110.

Depressurization of the sterilizing chamber 110 by the depressurizing subsystem 130 causes liquid on and in the device(s) 120 to gasify (e.g., evaporate, vaporize, etc.). It can also engender the release of sterilant from the sterilizing subassembly 170. For example, liquid inside the device(s) 120 can become vaporized and can escape from various ports and other non-sealed portions of the housing. The sterilant from the sterilizing subassembly 170 can follow the same path into the device, thus flooding the device with a desired amount of sterilant. Evaporation of the liquid away from the device(s) 120 is an endothermic process (i.e., involving latent heat) that causes a temperature drop in the sterilizing chamber 110 around the device(s) 120. In some implementations, this can frustrate (e.g., slow) the sterilizing process. Accordingly, some embodiments add heat to the sterilizing chamber 110. In some implementations, the amount of heat added to the environment is only as much as sufficient to overcome the latent heat of vaporization. In other implementations, other amounts of heat are provided to the environment within the sterilizing chamber 110. For example, additional heat can be added to activate and/or speed up the sterilizing process, or heat can be added in varying amounts over time for various purposes. For example, the amount of heat (e.g., and/or a profile of changes in temperature and/or pressure over time) can be tailored to particular implementations of encapsulated sterilants and corresponding vapor pressures for release of those sterilants.

Embodiments described herein use conductive heat to provide heating to the device(s) 120 within the sterilizing chamber 110. A heating subsystem 140 heats a conductive thermal assembly 115 (e.g., and the sterilizing subassembly 170 in some implementations), which is in contact with the device(s) 120 and is configured to conduct heat to the device(s) 120. Implementations of the conductive thermal assembly 115 at least partially conform to an external shape of the device(s) 120 so as to at least partially surround the portable electronic medical device 120. For example, the conductive thermal assembly 115 can be designed so that the portable electronic medical device, non-electronic medical device, instrument or other 120 is gently immersed in, sandwiched between, or otherwise in conformed contact with elements of the conductive thermal assembly 115. For example, conductive beads, heat packs, etc. can be assembled in a manner that dynamically conform to the geometry of one or more types of portable electronic medical devices, non-electronic medical devices, etc. when such device(s) 120 are moved into contact with the conductive thermal assembly 115. Examples of various conformable conductive thermal assemblies are set forth in co-owned U.S. Pat. No. 8,689,461, the entire contents of which are incorporated herein by reference.

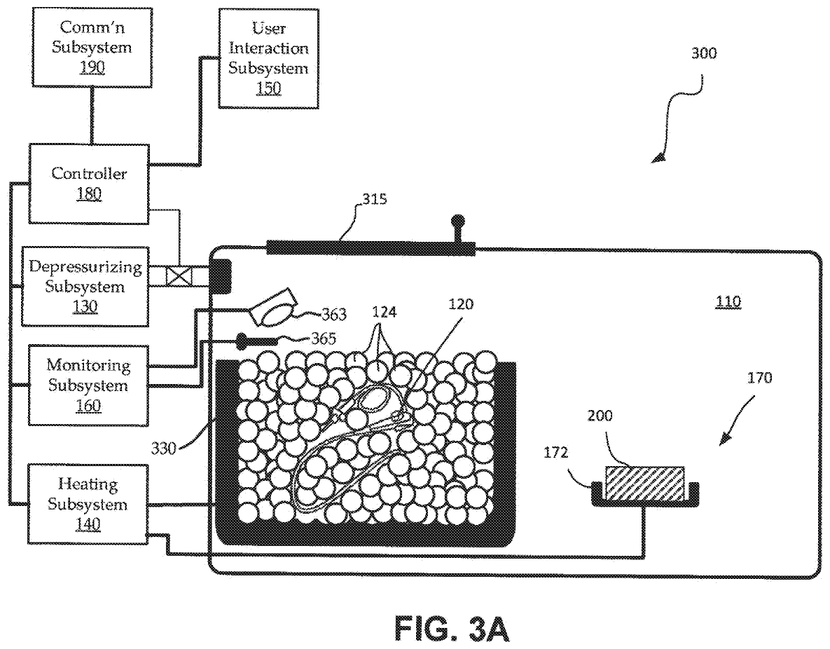

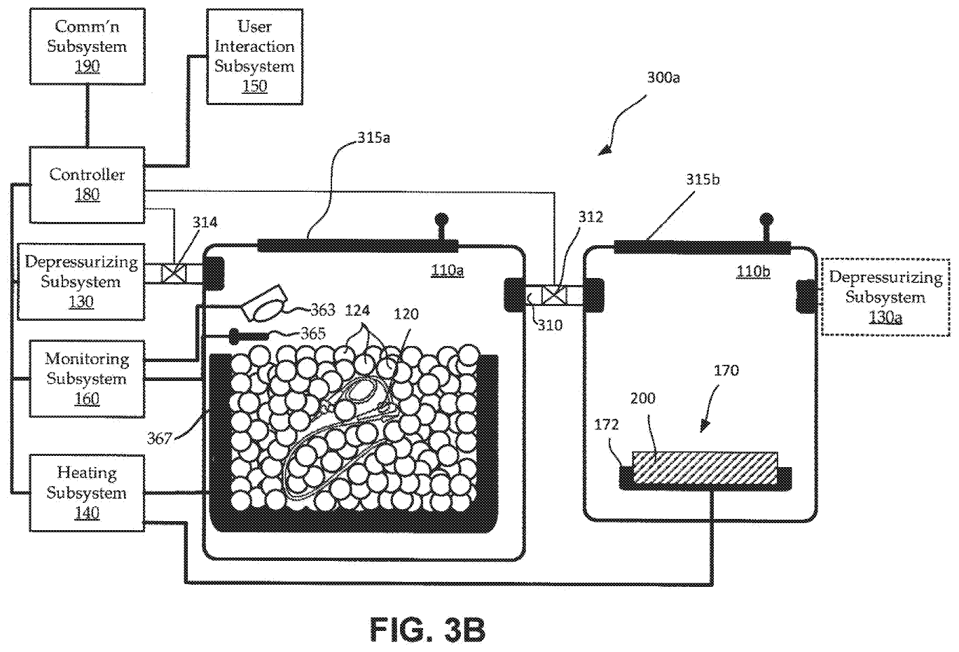

As shown in FIG. 3A, one implementation of the conductive thermal assembly 115 includes a number of thermally conductive beads. For example, a portion of the sterilizing chamber 110 is partially filled with small aluminum spheres or other conductive beads (e.g., zeolites), which need not be spheres, sized to be small enough to substantially conform to the shape of the device(s) 120 when the device(s) 120 are placed in the beads (e.g., partially or fully submerged into the bed of beads). Of note, the beads 124 and/or the medical device are not to scale. The spheres or beads (hereafter beads) 124 are also sized to be larger than any port or opening in the device(s) 120. In such an implementation, the heating subsystem 140 can heat the sterilizing chamber 110 from the outside (e.g., from the bottom and/or sides of the sterilizing chamber 110). The applied heat from the heating subsystem 140 (e.g., resistive electrical heater or radiant heater that heats the beads) is conducted toward the device(s) 120 via the beads, permitting the heat to evenly and gently surround at least a portion of the device(s) 120.

Experimentation by the inventors has demonstrated that the beads tend to store heat in their mass, so that cooling from the latent heat of vaporization can be counteracted by heat stored in the beads adjacent to the device(s) 120. Some implementations select beads having relatively high thermal capacity (e.g., storage), which can tend to provide a steady flow of heat to the device(s) 120 without exceeding maximum temperature limits. For example, beads with low thermal conductivity and/or low heat storage capacity can tend to allow cold regions to form around the device(s) 120 as the liquid gasifies, potentially quenching the gasification of the liquid once the temperature drops below a phase change temperature at that level of vacuum.

Returning to FIG. 1, other subsystems are used in some embodiments to provide additional functionality. Some embodiments include a monitoring subsystem 160 that can provide feedback control, environmental monitoring within the sterilizing chamber 110, monitoring of the device(s) 120, etc. Implementations of the monitoring subsystem 160 include one or more probes, sensors, cameras, and/or any other suitable device. In one embodiment, the monitoring subsystem 160 includes one or more sensors situated inside the sterilizing chamber 110 and configured to monitor internal pressure (vacuum level), humidity, temperature, and sterilization level within the sterilizing chamber 110. For example, the measurements can be used to determine if the heating is sufficient to overcome the latent heat of vaporization, to determine if the vacuum level is sufficient, to determine when the device(s) 120 has dried sufficiently, to determine if enough sterilant has been released to completely sterilize the component, etc.

The monitoring subsystem 160 can communicate its measurements through wired and/or wireless communications links to a controller 180 located outside the sterilizing chamber 110. For example, the controller 180 includes memory (e.g., non-transient, computer-readable memory) and a processor (e.g., implemented as one or more physical processors, one or more processor cores, etc.). The memory has instructions stored thereon, which, when executed, cause the processor to perform various functions. The functions can be informed by (e.g., directed by, modified according to, etc.) feedback from the monitoring subsystem 160. For example, the measurements from the monitoring subsystem 160 can be used to determine when to end the sterilizing routine and release a pressure release valve of the sterilizing chamber 110, when and how to modify the heat being delivered to the conductive thermal assembly 115, etc. The controller 180 can also direct operation of other subsystems, such as the conveyor assembly 125, pressurizing subsystem 130, etc.

In some embodiments, the monitoring subsystem 160 includes a camera 363 configured to “watch” the internal environment of the sterilizing chamber 110. In one implementation, the camera is used to monitor the vaporization of liquid from the device(s) 120. In another implementation, the camera uses infrared to indicate internal temperature readings from within the sterilizing chamber 110 and/or around the surface of the device(s) 120. In yet another implementation, the camera can monitor functionality of the device(s) 120 within the sterilizing chamber 110. For example, device(s) 120 may be plugged in within the sterilizing chamber 110, and a signal can be sent to the device(s) 120 (e.g., a monitoring message can be sent to the device(s) 120) within the sterilizing chamber 110 to see if the device(s) 120 react. The camera can be used to visually monitor the reaction to determine whether the device(s) 120 was unharmed. In some implementations, the camera is used for other functions, for example, to capture “before” imagery of the device(s) 120 to help determine whether the device(s) 120 had pre-existing conditions (e.g., a cracks etc.) prior to using the sterilizing system 105. The monitoring system 160 can further include one or more sensors 365 (e.g., temperature, humidity, etc.).

Some embodiments of the sterilizing system 105 further include a user interaction subsystem 150 that facilitates user 103 interaction with functions of the system (e.g., using one or more displays, interface devices, quality interfaces, etc.).

Referring again to FIG. 3A, an embodiment of a sterilizing system 300, which may be utilized in a medical facility, is shown. The sterilizing system 300 can be a non-limiting embodiment of sterilizing system 105 of FIG. 1, and its components are described using the same reference numbers, where appropriate, for the sake of added clarity. The sterilizing system 300 is designed to receive device(s) 120 into the sterilizing chamber 110 via a door 315. For example, the door 315 may be disposed on a top surface of the chamber 110 and includes any gaskets or other seals to allow the sterilizing chamber 110 to be sufficiently sealed when the door 315 is closed and the sterilizing chamber 110 is pressurized. A similar form factor can be designed to support multiple sterilizing chambers 110 for concurrent sterilizing (and/or disinfecting) of multiple device(s) 120 and/or for sterilizing of multiple types of device(s) 120.

The sterilizing chamber 110 is pressurized by a pressurizing subsystem 130 (e.g., a vacuum pump or the like in fluid communication with the sterilizing chamber 110 via suitable hoses, seals, valves, etc.). A heating subsystem 140 is coupled with the sterilizing chamber 110 in such a way as to provide heat to a conductive thermal assembly 330 and sterilizing subassembly 170 inside the sterilizing chamber 110. As illustrated, the conductive thermal assembly 115 can include a number of thermally conductive beads 124 supported on or within a first resistive heating element 330. Operation of the heating element 330 heats the thermally conductive beads. In one arrangement, the beads 124 may be coated with a solid-form sterilant. In such an arrangement, the beads define the sterilizing subassembly. In the illustrated embodiment, the sterilizing subassembly is formed of a separate package 200 of solid-form sterilant that may be disposed within the chamber 110. The sterilizing subassembly may further include a second heater 172 for separately heating the package 200 of sterilant. In the illustrated embodiment, the second heater 172 is a second resistive heating element 172 that may support and conductively heat the sterilant package 200. The second heater 172 is controlled by the heating subsystem 140. It will be appreciated that the second heater may take alternate forms (e.g., a radiant heater focused on the package, etc.). In any embodiment, the medical device 120 and the sterilant package 200 may be heated to different temperatures. The sterilizing chamber 110 is configured to receive the device(s) 120 in a position that allows the beads of the conductive thermal assembly 115 to substantially conform to at least a portion of the device(s) 120 geometry and to conduct heat to the device(s) 120. In an arrangement where the beads are coated with a solid form sterilant, heating of the beads in conjunction with reducing pressure in the chamber allows the sterilant that is encased in the polymer or other matrix to be released, thus sterilizing the device(s) 120. In an arrangement where the separate sterilant package 200 is provided, heating of the package 200 by the second heater 172 in conjunction with reducing pressure in the chamber 110 allows the sterilant that is encased in the package to be released.

FIG. 3B illustrates another embodiment of a sterilizing system 300a. The sterilizing system 300a and its components are described using the same reference numbers as the embodiment of the sterilizing system 300 of FIG. 3A, where appropriate. This embodiment of the sterilizing system 300a is substantially similar to the sterilizing system 300 of FIG. 3A with the exception that this system 300a utilizes first and second separate chambers 110a and 110b. In this regard, the first chamber 110a (e.g., primary chamber) may include a conductive thermal assembly 367 configured to receive one or more medical devices 120. The second chamber 110b (e.g., antechamber) houses the sterilant subassembly 170. As shown, the illustrated sterilant subassembly 170 again includes a conductive heater 172 that supports a package 200 of solid-form sterilant (e.g., liquid sterilant disposed within a matrix). The heater 172 may heat the package 200 to a desired temperature within the second chamber 110b. The second chamber 110b may include a second door 315b, which allows a user to place the solid form sterilant therein.

In this embodiment, the first and second chambers 110a and 110b are in selective fluid communication. That is, these chambers 110a, 110b connected via a fluid paths or conduit 310. In the present embodiment, the conduit 310 further includes valve 312 that may be actuated/controlled by the controller 180. Each of the illustrated embodiments includes a second valve 314 between the first chamber 110a and the pressurizing subsystem (e.g., vacuum pump). Again, this valve 314 may be controlled by the controller 118. In operation, both valves 312, 314 may be opened to permit the pressurizing subsystem 130 to evacuate each chamber 110a, 110b. In various operations, upon achieving a desired pressure level (e.g., vacuum) the first valve 312 may be close to permit off-gassing of the sterilant from the sterilant package 200 into the second chamber 110b. The second valve 314 may remain open to permit continued evaporation and removal of water or other liquids from the medical device 120. When desired, the first valve 312 may be opened to permit adiabatic expansion of the gasified sterilant into the first chamber 110a. Additionally, or optionally, the second valve 314 may be closed after water or other liquids are evaporated from the medical device and prior to opening the first valve to allow sterilant to enter into the main chamber 110a. In any case, such an arrangement permits adiabatic expansion of the gasified sterilant into the first chamber and into the medical device such that gasified sterilant is able to expand into all evacuated interior areas of the medical device. Such an arrangement may allow for reduced use of sterilant compared to a system that continually draws vacuum. Though illustrated as utilizing a single pressurizing system 130 for both chambers 110, 110b, it will be appreciated that the second chamber may have a separate pressurizing system (e.g., 130a). Though discussed in relation to fluidly isolating the chambers, it will be appreciated that in some implementations, the chambers may remain in fluid communication throughout the process. In this implementation, the second chamber may primarily be used to control the separate heating of the solid-form sterilant.

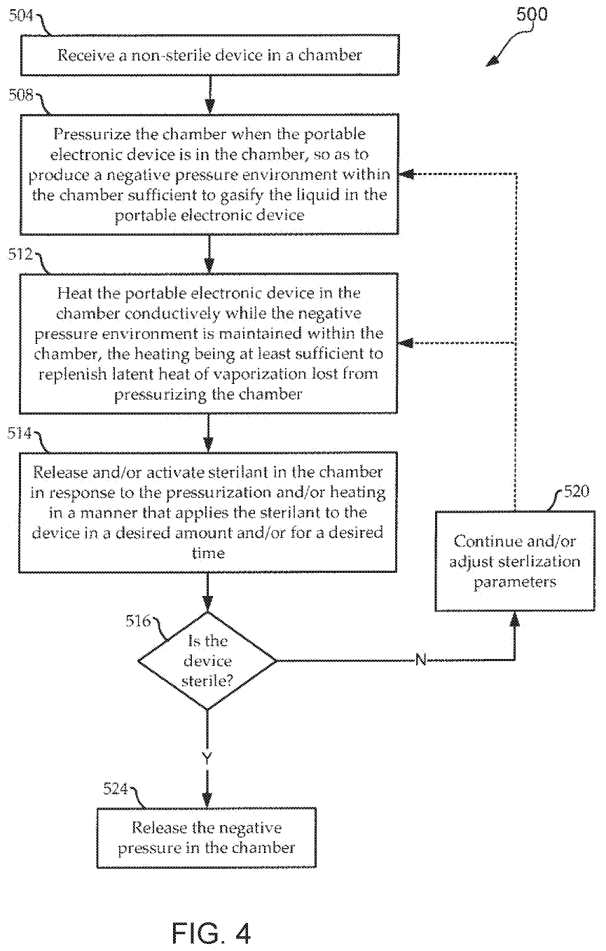

FIG. 4 shows a flow diagram of one illustrative method 500 for sterilizing an electronic medical device, non-electronic medical devices, instruments and other medical items 120, according to various embodiments. The method 500 operates in context of sterilizing systems, such as those described above with reference to FIGS. 1-3. Embodiments begin at stage 504, by receiving a device 120 (e.g., a portable electronic medical device, non-electronic medical device, instrument, etc.) in a chamber. As described above, it is assumed that the device is non-sterile. For example, the device is scheduled for sterilization, assumed to need sterilization (e.g., after a procedure, in accordance with a policy or standard, etc.), the device is known to have an excessive amount of contamination in (and possibly on) the device, etc. The device can be placed in the chamber through a door or other scalable opening in the chamber. Typically, the device is placed into contact with a thermal conduction assembly or the device and/or the thermal conduction assembly are moved into contact with each other as the method 500 begins (e.g., when the chamber door is closed, etc.).

At stage 508, the chamber is pressurized when the device(s) 120 are in the chamber, so as to produce a negative pressure environment (e.g., a substantial vacuum) within the chamber sufficient to gasify the liquid in the device(s) 120. For example, the chamber is fluidly coupled with a vacuum pump. When the vacuum is established and the liquid gasifies, latent heat of vaporization is lost.

At stage 512, the device(s) 120 are conductively heated in the chamber (e.g., via a thermal conduction assembly, such as beads) while the negative pressure environment is maintained within the chamber. The heating is at least sufficient to replenish the latent heat of vaporization lost from pressurizing the chamber. As described above, any suitable type of thermal conduction assembly can be used. Further the thermal conduction assembly may at least partially conform to an external shape of the device(s) 120.

At stage 514, pressurization at stage 508 and/or heating at stage 512 causes sterilant in the chamber to be released and/or activated in a manner that applies the sterilant to the device(s) 120 in a desired amount and/or for a desired time. For example, the pressurization at stage 508 causes vaporization or off-gassing of encapsulated liquid sterilant in a package within the chamber.

In some implementations, at stage 516, a determination is made as to whether the excess contamination has been removed from the device(s) 120 (e.g., whether the device is sufficiently sterile). If not, one or more steps can be taken, such as maintaining and/or adjusting the sterilization parameters (e.g., negative pressure and/or the heating within the chamber). If so, at stage 524, the negative pressure in the chamber can be released (e.g., via a release valve).

The process illustrated in FIG. 4 can be employed for sanitation. It will be recognized by one or ordinary skill in the art that process pressures, times and temperatures may be different for sanitation compared to sterilization.

In embodiments, a sterilant is any a solid, liquid and/or vapor, which provides sanitation or sterilization. In particular embodiments, a preferred sterilant exhibits improved volatilization and molecular transport in a sub-atmospheric environment as a result of being in composition with an MME. In specific embodiments, the vapor pressure of the sterilant at the operating temperature of the apparatus is greater than or equal to 10 torr. In specific embodiments, the vapor pressure of the sterilant at the operating temperature of the apparatus is greater than or equal to 100 torr. In embodiments, the sterilant is a liquid at NPT. In an embodiment, sterilant vapor is intended to contact substantially all surfaces of the article including any internal surfaces of the article, such as those associated with a crevice, notch, hole, indentation, channel, lumen or the like. Exemplary sterilants include hydrogen peroxide (H₂O₂), peroxyacids (peracetic, performic, etc.), alcohols (e.g. isopropanol, methanol, etc.). Additional examples of sterilants and forms of mixtures of sterilant and MME are provided in U.S. provisional applications, Attorney Docket No. 3-20P US and Attorney Docket No. 10-20P US, filed Jan. 28, 2020. Each of these applications are incorporated by reference herein in its entirety.

The sterilant, in an embodiment, may initially be stabilized in a matrix material where the sterilant molecules are reversibly trapped in the interior of the matrix to prevent reaction and to suppress degradation. In an embodiment, a Molecular Mobility Enhancer (MME) may also be trapped in the interior of the matrix. The stabilization matrix may consist of synthetic polymers, biopolymers, ceramics, glass, or composite materials, or any combination of these materials. Some examples of synthetic polymers that may be used include, but are not limited to, silicones, polyacrylates, polyethylenes and related polymers, polyamides, polyurethanes, polyethers, polyphosphazenes, polyanhydrides, polyacetals, poly (ortho esters), polyphosphoesters, polycaprolactones, polylactides, and polycarbonates. Some examples of biopolymers that may be used include, but are not limited to, carbohydrates, starches, celluloses, chitosan's, chitins, dextran's, gelatins, lignin's and polyamine acids. Some examples of ceramics include, but are not limited to, aluminum oxide, zirconium oxide, silicon dioxide, magnesium oxide, titanium oxide, aluminum nitride, silicon nitride, boron nitride and silicon carbide. Composite materials may consist, but are not limited to, two or more of the matrix materials listed above.

The sterilant is typically prepared in liquid form (e.g., in situ) and then mixed or otherwise loaded into the stabilization matrix. The sterilant matrix is used to form a gel or a solid that contains the sterilant molecules. The sterilant gel or solid may be shaped into the form of beads, a block and/or may be stored in a container or wrapping that is sufficiently porous and such that allows the sterilant vapor molecules to outgas from the material matrix and/or container when exposed to reduced pressure and/or increased temperature. The purity and concentration of sterilant loaded into the matrix material can be adjusted according to the intended use. It may be seen that a sterilant gel or solid that contains a purer and more concentrated form of sterilant may be used for multiple sterilization runs, for killing more resistant microbes, and/or for sterilizing a larger bioload. The sterilizing system can be configured for a user to easily and safely place the sterilant gel or solid into the chamber before pressurizing the system and prior to initiating a sterilization cycle. The encapsulation of sterilant in a matrix material may also provide a simplified method for extending the shelf-life of the sterilant in such a way that the sterilant does not need to be prepared just prior to use. The encapsulated sterilant may sit at or near temperatures without spontaneously degrading for an extended period giving the user the opportunity to pull the sterilant gel or solid off of the self when needed to insert it into the sterilizing system for one or more uses.

As noted, the encapsulated sterilant (e.g., solid-form sterilant) may be provided in a container or wrapper. In one implementation, the sterilant may be provided in a gas permeable packet. In such an implementation, the solid-form sterilant may be provided in, for example vapor gas porous polytetrafluoroethylene (PTFE), polyethersulfone (PES) or high-density polyethylene (HDPE) membranes, to name a few. The exact gas-permeable membrane selected may be based on the sterilant and/or matrix material of a given solid-form sterilant. In some implementations, the solid-form sterilant may be disposed within a sealed gas-permeable membrane and then sealed within a non-permeable wrapper. Prior to use, the non-permeable wrapper may be removed and the solid-form sterilant within the gas permeable membrane may be inserted into the chamber or antechamber of the sterilizing system. Alternatively, the solid-form matrix may be removed from a non-permeable container or wrapper and placed directly within a sterilizing system.

In various implementations, it may be desirable to heat the solid-form sterilant to a temperature that is elevated compared to a temperature that a medical device is heated (e.g. to replace the latent heat of vaporization lost during the pressurization. For instance, many electrical devices may degrade at temperatures above about 30 degrees Celsius. However, depending on the matrix material and sterilant, a solid-form sterilant may require or better vaporize (e.g., off-gas) at higher temperatures. Accordingly, by providing separate heaters and/or chambers, the temperatures of the medical device and sterilant may be individually controlled. This may provide improved sterilizing performance.

The use of the solid-form sterilant was found to provide significantly improved performance in comparison with the use of liquid sterilants. It is believed that liquid sterilants evaporate too quickly in negative pressure environments. Along these lines, it was determined that use of liquid sterilants, to achieve substantially equal levels of sterilization, required a significant increase in the amount of sterilant utilized, which, in some instances raised concerns about corrosion.

Vapor generated under reduced pressure in apparatus, enclosures or processing chambers herein is employed to sterilize the apparatus, enclosures or processing chambers and more particularly any articles therein (substrates, implements, devices, instruments etc.) In an embodiment, the article to be sterilized comprises one or more surface to be treated which is diffusion restricted. The phrase “diffusion restricted”, in reference to the apparatus and method described in the disclosure, is understood to mean, but is not limited to, an object or area on or within an object that contains a material and/or configuration such that the physical and/or chemical properties of said material and/or configuration retards or slows the rate at which the movement of anything (e.g. vapor molecules, etc.) can move through the said material and/or configuration of the said object and/or area on or within the article. In embodiments, the vapor is generated in the apparatus at ambient room temperature such that the apparatus is not heated. In embodiments, the vapor is generated in the apparatus (enclosure etc.) at a selected temperature above ambient room temperature. In embodiments, the selected temperature ranges from above ambient room temperature to 100° C. In embodiments, the selected temperature ranges from above ambient room temperature to 50° C. In addition, mixtures of sterilant and MME, particularly those in solid form, may be separately heated from the apparatus or enclosure. Such mixtures may be separately heated from ambient room temperature up to temperatures up to 150° C. or more preferably up to temperatures of 100° C.

Different portions of the system may be operated at different temperatures. For example, the process chamber may be operated at one temperature and other portions of the system may be operated at temperature(s) different from the processing chamber. The process chamber which contains vapor comprising the transport moiety or a mixture of MME and transport moiety may be operated at ambient room temperature or heated (by any known means) to a higher temperature up to 100° C. Preferably, the process chamber is operated at a temperature ranging from ambient room temperature to 60° C.

The pressure in the process chamber is preferably maintained at a selected pressure for a selected time to initiate and complete on outgassing and conversion of the MME and/or transport moiety into vapor. One skilled in the art would understand that the holding vapor chamber pressure and the vapor pressure holding time can be adapted to a given application of the MME-transport moiety blend vapor. In embodiments, the selected pressure maintained in the apparatus ranges from 0.1 torr to 200 torr. More specifically, the selected pressure maintained in the apparatus ranges from 0.5 torr to 10 torr. More specifically, the selected holding pressure maintained in the apparatus is about 0.5 to 3 torr. Pressure is generally maintained at the selected value+/−10%. In embodiments, the selected holding pressure time is maintained for 1 minute to 24 hours. In embodiments, the selected holding pressure time is maintained for 5 minutes to 1 hour. In embodiments, the selected holding pressure time is 5 minutes to 30 minutes. In a specific embodiment, the selected holding pressure time is 15 minutes+/−10%.

### Embodiments of the Sterilant

### Peri-Peroxy Acid Solution

According to an embodiment, a peri-peroxy acid solution can be generated in a way that compliments the need of the user. For example, the formulation of the peri-peroxy acid solution can be adjusted according to its intended use. It may contain a more pure and concentrated solution if it is to be used for multiple sterilization runs, for killing more resistant microbes and/or for sterilizing a larger bioload. As such, peri-peroxy acid solution chemistries described herein are meant to include mixed peri-peroxy acid chemistries. The formulation methods described may or may not utilize a catalyst to accelerate the formation of the peri-peroxy acid. Additionally, the formulation methods described may or may not utilize a stabilizer to prevent reaction and subsequent decomposition enhancing the shelf-life and safety of the sterilant solution. The selective production of peri-peroxy acid can be achieved by controlling the type and proportions of component chemicals as well as reaction conditions, such as temperature and rate of mixing. The average peroxy acid content is preferably between 0.5 and 20 wt % while the average MME content is preferably between 0.1 and 5 wt %, however the average concentration of these components may vary depending on reaction condition variables. In some embodiments, the peri-peroxy acid compositions may be generated from a mixture of more than one peroxy acid with/or without an MME.

### Peroxy Acid Components

In one embodiment, the peroxy acid can be prepared by mixing one or more carboxylic acids followed by the addition of a hydrogen peroxide solution. A preferable concentration of carboxylic acid being introduced into the reaction mixture may be between 25 to 100 wt %. The hydrogen peroxide concentration may be between 1 and 90 wt % and may be introduced into the reaction mixture at a preferable concentration between 5 and 60 wt %. The carboxylic acids used for formation of the peroxy acid components may consist of an aliphatic carboxylic acid that may include but is not limited to acetic acid, formic acid, propionic acid, phtalic acid, oxalic acid, malic acid, maleic acid and fumaric acid or mixtures of such. The carboxylic acids used for formation of the peroxy acid components may also consist of an aromatic carboxylic acid that may include but is not limited to benzoic acid, salicyclic acid, gallic acid, toluic acid phthalic acid, isophthalic acid and teraphthalic acid.

In another embodiment, the peroxy acid can be generated by reacting an alternative carboxlic acid source with a reactive oxidizing species. The alternative carboxylic acid source may consist of a liquid or a solid and may be used in the methods described in this invention. Any suitable substance that is a peroxy acid generator may be used. The peroxy acid source may consist of one or more salt of the desired carboxylic acid, for example, the salt of acetic acid, such as an acetate, e.g. sodium or ammonium acetate. Alternatively, a carboxylic ester, e.g. ethyl acetate, propyl acetate, dibutyl phthalate, etc., may be used as an alternative carboxylic acid source. The reactive oxidizing species may include hydrogen peroxide or hydrogen peroxide derivative that delivers a hydrogen peroxide concentration between 1 and 90 wt % of hydrogen peroxide. Suitable species include urea-hydrogen peroxide adducts or hydrogen peroxide donors including but not limited to lithium peroxide, sodium peroxide, magnesium peroxide, calcium peroxide, strontium peroxide, barium peroxide, zinc peroxide. Other examples include sodium perborate tetra-, tri- or mono-hydrate, persilicates, peroxycarbonates, peroxynitrous acid or its salts, peroxyphosphoric acid or its salts, peroxysulfuric acid or its salts, sodium periodate, potassium perchlorate, and/or any transition metal peroxides or any other source of peroxide such as polymer supported peroxides.

The same may be reacted in the presence of a catalyst in order to accelerate the formation of performic acid. Commonly used catalysts include but are not limited to mineral acids such as sulfuric acid, sulfonic acid, methanesulfonic acid, nitric acid, phosphonic acid, phosphoric acid, pyrophosphoric acid and/or polyphosphoric acid. Other non-limiting examples includes molecules that contain at least one ester group, such as carboxylic esters such as caproic acid mono- and di-glycerides, 1,2,3-triacetoxypropane, ethyl acetate, ethyl propionate, methyl formate, sorbitan monolaurate, dibuty phthalate, glycol acid buty ester and/or any sugar ester. Other catalytic ester compounds include, but are not limited to, sulfate esters, sulfonate ester or lactone and/or phosphate esters.

The type and amount of catalyst that may be added to the composition or composition components is not limited in particular. One or more catalysts can be added to the reaction mixture in a concentration ranging from between 0.01 to 20% by weight and is dependent on the mass of the peri-peroxyacid. The catalyst may be added to a peri-peroxy acid mixture. Alternatively, the catalyst may be dissolved independently in either the hydrogen peroxide, or the carboxylic acid before mixing. Or, the catalyst may be dissolved in another suitable solvent before adding to the mixture or mixture components.

In some embodiments a stabilizing agent is preferred for certain peri-peroxy acid compositions. In such cases, one or more stabilizers may be added to prevent reaction and subsequent decomposition all while enhancing the shelf-life and safety of the peri-peroxy acid solution. Stabilizers are selected that do not adversely alter the vapor pressure or the purity of the active ingredient in the sterilant solution. Equally important, stabilizers are selected such that they may not counteract the vaporization properties and the anti-microbial and/or anti-sporicidal properties of the peri-peroxy acid. One or more agents can be used and may include organic amino- or hydroxyl-polyphosphonic acid or soluble salt, polymeric carboxylic acid or soluble salt, hydroxycarboxylic acids, aminocarboxylic acids, heterocyclic carboxylic acid (e.g. dipicolinic acid), 1-hydroxy ethylidene-1,1-diphosphonic acid (HEDP), other phosphonic acids and phosphonate salts such as ethylenediamine tetrakis methylenephosphonic acid (EDTMP), diethylenetriamine pentakis methylenephosphonic acid (DTPMP), cyclohexane-1,2-tetramethylene phosphonic acid, amino[tri(methylene phosphonic acid)], (ethylene diamine[tetra methylene-phosphoic acid)], 2-phosphene butane-1,2-4-tricarboxylic acid, other salts such as the alkali metal salts (e.g. ammonium or alkyloyl amine salts), mono-, di-, or tetra-ethanolamine salts, piconlinic acid or combinations thereof. The stabilizer may be added to a mixture of peri-peroxy acid or a mixture of peri-peroxy acid containing a catalyst. Alternatively, the stabilizer may be dissolved independently in either the hydrogen peroxide, the peroxy acid or the catalyst before mixing. Or, the stabilizer may be dissolved in another suitable solvent before adding to the mixture or mixture components. The type and amount of stabilizer that may be added to the composition or composition components is not limited in particular, but may be between 0.01% and 5% by weight and is dependent on the mass of the peri-peroxyacid.

### MME Components

The sterilant composition, particularly hydrogen peroxide or peri-peroxy acid solution, may also include MME agents or components.

The term “molecular mobility enhancer (MME)” refers to a chemical component of a mixture whose volatilization and mechanism of transport properties are sufficient to expedite the release of molecules, particularly transport moieties as described herein, from solids and to improve the movement of vapor molecules in a sub-atmospheric pressure environment by preventing molecular aggregation and/or stagnation. Some non-limiting examples of MMEs include any volatile alcohol (e.g. methanol, ethanol, isopropanol, etc.), alkane (e.g. pentane, hexane, heptane, etc.), carboxylic acid (e.g. formic acid, acetic acid, propionic acid, etc.), ester (e.g. ethyl acetate, isopentyl acetate, etc.), ether (e.g. diethyl ether, methyl phenyl ether, tetrahydrofuran, etc.), ketone (e.g. acetone, diacetyl, cyclobutanone, etc.), etc. Additional examples of MMEs are provided in U.S. provisional applications Attorney Docket NO. 3-20P US and Attorney Docket No: 10-20P, both filed Jan. 28, 20120.

In embodiments, the MME moiety has a vapor pressure of greater than or equal to 10 torr and more preferably greater than or equal to 100 torr at the operating temperature of the apparatus. In more specific embodiments, the vapor pressure of the MME is 20 torr or higher, 30 torr or higher or 40 torr or higher at the operating temperatures of the apparatus.

In embodiments, the MME is a liquid or a solid at normal temperature and pressure (20° C. and 1 atm (760 torr). In embodiments, the vapor pressure of the MME is greater than or equal to the vapor pressure of the transport moiety at the apparatus operating temperatures. In embodiments, the vapor pressure of the MME may also be less than or equal to the vapor pressure of the transport moiety at the apparatus operating temperatures. For the purpose of this disclosure, the MME must have a vapor pressure that increases the vapor pressure of the transport moiety upon blending at a concentration such that the vapor-liquid ratio of the blend at room temperature and at 10 Torr is between 1 and 90 wt %, inclusive, more preferably less than or equal to 50 wt %. The MME-transport moiety blend with a vapor-liquid ratio under the conditions described above would favorably change the vapor pressure of the desired transport moiety such that the transport moiety would vaporize faster and at lower temperatures.

Generally, an MME with a higher vapor pressure should be added to the transport moiety to increase the vapor pressure of the transport moiety but, in some implementations, an MME with a lower vapor pressure may also be added to the transport moiety so as to also advantageously affect vaporization kinetics and vapor pressure of the transport moiety. Generally speaking, a lower vapor pressure MME, when added to a transport moiety may readily break the attractive forces of the transport moiety molecules effectively causing the transport moiety to readily evaporate ultimately raising the vapor pressure of the transport moiety. One skilled in the art would understand that the temperature, pressure and/or vapor-liquid ratio of the MME-transport moiety blend can be adjusted to complement desired MME-transport moiety properties and/or process conditions.

The MME is a component whose volatilization and mechanism of transport properties are sufficient to carry out the methods of this disclosure. The volatilization properties of an MME not only expedite the release of molecules from liquid or solids but also improve the movement of vapor molecules in a sub-atmospheric pressure environment by preventing molecular aggregation and/or stagnation. MME vapor helps by negating the effects of transport moiety aggregation and/or stagnation by providing a transport conduit by which the transport moiety vapor experiences increased velocity, improved laminar flow and deliverability to target surfaces. Improvement in mobility of transport moiety vapor to a target surface makes it possible to direct the flow of the desired gaseous material into diffusion restricted areas. MME gaseous material serves to prevent irregularities in the pattern of vapor flow at different parts of an object or a device by enabling the user to control the speed at which a transport moiety vapor passes over a material. It may be preferable to decrease or increase the rate of vapor exposure depending on the nature of the transport moiety vapor. For example, if a user was employing a more reactive and mobile sterilant, such as performic acid, it may be advantageous to increase the rate of vapor exposure. It should be noted that the rate of vapor exposure can be controlled by adjusting the MME-transport moiety blend as well as process condition.

The MME may be selected from, but is not limited to, one or more of any alcohol, any alkane, any carboxylic acid, any ester, any ether and/or any ketone or any combination thereof. Alcohols may include, but are not limited to, any linear, branched, cyclic, primary, secondary, tertiary alcohol, polyol and/or isomeric form of a C1-C20 alcohol or in more specific embodiments a C1-C12, a C1-C6 or a C1-C4 alcohol. Some examples may include methanol, ethanol, isopropanol, etc. All combinations and subcombinations of alcohols are included (e.g. alcohols that comprise mixtures such as ethanol and isopropanol).

Alkanes may include, but are not limited to, any linear, branched, cyclic, saturated, unsaturated, polymeric and/or isomeric form of a C5-C20 alkane, or in more specific embodiments a C5-C10 alkane. Some examples may include pentane, hexane, heptane, etc. All combinations and subcombinations of alkanes are included (e.g. alkanes that comprise mixtures such as pentane and hexane, etc.).

Carboxylic acid may include, but are not limited to, any linear, branched, cyclic, saturated, unsaturated, polycarboxylic acid, hydroxy and keto acid and/or any amino acid having 1-20 carbon atoms, or in more specific embodiments, those having 1-12 carbon atoms, those having 1-6 carbon atoms or those having 1-3 carbon atoms. Some examples may include formic acid, acetic acid, propionic acid. All combinations and subcombinations of carboxylic acids are included (e.g. carboxylic acids that comprise mixtures such as acetic acid and citric acid).

Esters may include, but are not limited to, any linear, branched, cyclic, saturated, unsaturated, poly-ester, and/or isomeric form of a C3-C20 ester, or in more specific embodiments, C3-C12 esters, C3-C8 esters or C3 to C6 esters. Some examples may include ethyl acetate, methyl butyrate, methyl anthranilate. All combinations and subcombinations of esters are included (e.g. esters that comprise mixtures such as ethyl acetate and isopentyl acetate).

Ethers may include, but are not limited to, any linear, branched and/or cyclic, saturated, unsaturated, molecules containing multiple ether groups, and/or isomeric forms of a C4-C20 ether or in more specific embodiments C4-C12 ethers or C4-C8 ethers. Some examples may include diethyl ether, methyl phenyl ether, tetrahydrofuran, etc. All combinations and subcombinations of ethers are included (e.g. ethers that comprise mixtures such as cyclopropyl methyl ether and 1,4-dioxane).

Ketones may include, but are not limited to, linear, branched, cyclic, saturated, unsaturated, polyketones (e.g. acetyl, dimedone, etc.), and/or isomeric forms of a C3-C20 ketone or in specific embodiments C3-C12, C3-C8 or C3 to C6 ketones. Some examples may include acetone, diacetyl, cyclobutanone, etc. All combinations and subcombinations of ketones are included (e.g. ketones that comprise mixtures such as cyclopropenone and cyclobutanone, etc.)

The list of MMEs described should not be limiting as one skilled in the art, based on the knowledge of compounds having similar properties and/or activities in the apparatus described herein, may select other molecules to carry out the described methods of the disclosure. Additional sources of MME may include any aldehyde, alkene, alkyne, amide, amine, aniline, aromatic compound, halogen containing compound, nitriles, other nitrogen containing compound, phenol, thiol, sulfide, etc. The MME may also include any structural analog or structural derivative of a described compound such that any component or combination of components are sufficiently volatile under the conditions described in the disclosure to carry out the functions of the disclosure. A “structural analog” or “structural derivative” described herein may be defined as a compound with a structure that is similar to that of an alternative compound that differs from it with respect to a certain component. It may vary with respect to one or more atoms, functional groups or substructures which are replaced with alternative atoms, functional groups or substructures. For examples, a structural analog of methanol may include silanol.

Alternatively, an MME may be generated in situ prior to blending with the transport moiety. Any suitable alternative source that when in contact with a solution of solvent, such as water, may be used to generate the MME in situ may be used for the present method. For example, an alternative source may be a salt of an alkoxide, e.g. sodium ethoxide, which in the presence of water generates the alcohol ethanol. In another example, an alternative source may be a salt of a carboxylic acid, e.g. sodium formate, which in the presence of water generates the carboxylic acid formic acid. It should be appreciated that while a limited number of methods for generating MMEs in situ from alternative sources have been described herein other methods and/or alternative sources may be suitably used to generate MMEs in situ.

Components suitable for use in the present invention include but are not limited to ethers, alcohols, phenols, esters or amides. For example, suitable MMEs for various embodiments are ethers, which are preferred for facilitating the vaporization of peroxy acid molecules from solution and improving deliverability of the same throughout the vacuum chamber. An MME may be added to achieve a concentration of between 0.5 and 25 wt %. Suitable ethers may include but are not limited to any linear or aromatic, symmetrical or unsymmetrical ether such as dimethyl ether, diethyl ether, methyl n-propyl ether, methyl phenyl ether, ethyl phenyl ether, heptyl phenyl ether, methyl isopropyl ether, phenyl isopentyl ether, 1,2-dimethyloxyethane or 2-ethoxy-1-dimethylcyclo hexane. Suitable alcohols may include but are not limited to any linear or aromatic, mono-, di-, or tri-hydric, and/or primary, secondary or tertiary alcohols such as methanol, ethanol, 1-propanol, 2-propanol, 1-butanol, 2-butanol, 2-methyl-1-propanol, 2-methyl-2-propanol, 1-pentanol, 3-methyl-1-butanol, 2,2-dimethyl-1-propanol, cyclopentanol, 1-hexanol, cyclohexanol, 1-heptanol, 1-octanol, 1-nonanol, 1-decanol, 2-propen-1-ol, phenylmethanol, diphenylmethanol, triphenylmethanol, 2-propen-1-ol or 3-methyl-1-butanol. Suitable esters may include but are not limited to organic esters such as methanoate, ethanoate, propanoate, 2-methylpropanoate, butanoate, pentanoate, hexanoate, benzanoate, heptanoate, salicylate, octanoate, nonanoate, cinnanoate, decanoate or inorganic esters such as phosphate ester or sulfate ester. The MME may be added to a peri-peroxy acid solution with or without a catalyst. Alternatively, the MME may be dissolved independently in either component of the peri-peroxide solution (e.g. the carboxylic acid, the hydrogen peroxide or the catalyst) before mixing or the MME may act as a solvent for any of the peri-peroxy acid components. The MME may also be dissolved in another suitable solvent before adding to the mixture or mixture components. An MME or combination of MMES may be selected in such a way that it does not adversely react with the chemical and/or physical properties and such that it does not adversely affect the anti-microbicidal and/or anti-sporicidal properties of the peri-peroxy acid liquid solution or vapor. The MMES in general has a vapor pressure 10 Torr and more preferably ≥100 Torr. In general, the MME should be present in the peri-peroxy acid solution in amount that is between 1 and 90 wt %, inclusive, more preferably at 50 wt %.

Ethers, particularly diethyl ether, and mixtures of ethers are exemplary effective molecular mobility enhancing agent components for the composition of the peri-peroxy acid vapor source. In an embodiment, MMEs include an ether or miscible mixtures of one or more ethers that are liquids at normal temperature and pressure (NTP, 20° C. and an absolute pressure of 1 atmosphere). Other effective molecular mobility enhancing agents include, but are not limited to, any number of alcohols, phenols, esters and/or amides, so long as the MMEs being utilized do not readily react with the peroxy acid in the liquid and/or vapor form and are adequately volatilized to form a vapor of a concentration necessary to facilitate evaporation of the peroxy acid molecules out of a liquid or solid and to modulate transport of the peroxy acid vapor molecules in a desired capacity in a vacuum sterilization system as descried herein, with or without the assistance of heat at a temperature of equal to or less than what is required by the device being sterilized. In an embodiment, MME's include one or more ethers, alcohols, esters, phenols, and/or amides or any miscible mixture thereof that are liquids at NPT.

Some embodiments described herein use performic acid as the peroxy acid component of choice due to its high vapor pressure and tendency to volatilize rapidly, although there are others that will suffice. For example, performic acid has a higher vapor pressure (78 mmHg@25° C.) over its commonly used counterpart peracetic acid (14.5 mmHg@25° C.), making it more readily able to vaporize, allowing for an accelerated sterilization run. By the same token, performic acid is firstly vaporized from a mixture of its parent components, formic acid and aqueous hydrogen peroxide, making it an attractive choice for selectively excluding undesirable residual components, such as water vapor or formic acid, during a sterilization process. Other commonly used sterilants have several disadvantages. Hydrogen peroxide and ozone are stronger oxidizers than performic acid and, as such, can be corrosive to certain device materials being sterilized and may limit its use on some devices. Hydrogen peroxide vapor, for example, is incompatible with commonly used sterilization packaging materials containing cellulose as well as other items containing nylon. Furthermore, hydrogen peroxide vapor penetration capabilities are less than that of performic acid due to restricted movement, especially into small crevices, as a result of hydrogen peroxide vapor's molecular size, surface absorption characteristics and water vapor barrier properties. Additionally, performic acid is non-toxic, and there are no reports of tumorigenic properties. The bactericidal and sporicidal properties of peracetic acid, performic acid, and perpropionic acids have been documented, and of the three peroxy acids, performic acid activity as an anti-fungicide surpasses that of its counterparts. Performic acid's mode of action, as an active oxidizing agent for killing microbial cells, is a highly effective and fast acting process of cleaving disulfide bonds of microbial cells ultimately causing death of the cell. In addition to performic acid's highly effective kill mechanism, the sterilization action of performic acid is faster and has a lower concentration threshold than that of related peroxy acid compounds such as peracetic acid. The Association of Official Analytical Chemists (AOAC) completed a Sporicidal Challenge and found that the D-value of performic acid may be lower than five minutes at a low concentration of 1,800 ppm at 44° C.

Some examples of ethers include, but are not limited to, any number of or combination of linear or cyclic ethers. Ethers include those of formula R—O—R′, where R and R′ are, independently, straight-chain or branched alkyl groups and particularly C1-C6 straight chain or branched alkyl groups. Some examples of alcohols may include, but are not limited to, any number of or combination of linear or cycle mono-, di-, tri- or polyhydric alcohols that may also be primary, secondary or tertiary in nature. Some examples of esters may include, but are not limited to, any number of organic and/or inorganic ester. Some examples of amides may include, but are not limited to, any number or combination of linear or cyclic, primary, secondary and/or tertiary amide that which may be organic or inorganic.

Some embodiments described herein use as ether as the MME component of choice due to its desirable volatilization and mechanism of transport properties, although there are others that will suffice. For example, diethyl ether has a higher vapor pressure (520 mmHg@25° C.) than that of its solution component counterpart performic acid (78 mmHg@25° C.), making it a prime MME sterilant solution candidate for preventing molecular aggregation of performic acid molecules in solution and in vapor phase. The volatilization properties of an MME play an important role in the release of peroxy acid molecules from the peri-peroxy liquid solution and the movement of the peroxy acid molecules in the vacuum chamber under reduced pressure.

At a chosen temperature, more molecules that are in the vapor phase make it easier for future molecules to be released from solution into vapor form. As such, the MME component of the mixture will initiate release of a number of molecules into the vapor phase of the chamber, creating an environment that becomes conducive for the peroxy acid to begin forming and releasing vapor molecules from solution.

Additionally, the larger the number of MME component molecules in the vapor phase, the weaker the forces become between the molecules left in solution. The weaker the attractive forces, the lower the energy needed to release molecules from each other to form vapor. Once the MME and peroxy acid molecules are in vapor form the MME vapor molecules assist transport of the peroxy acid vapor molecules throughout the vacuum chamber during a sterilization run. MME vapor helps by negating the effects of condensation saturation and sterilant vapor molecule congregation and stagnation in undesirable areas of the chamber by increasing the velocity and improving the deliverability of the peroxy acid vapor molecules. The combined characteristics of the MME and peroxy acid sterilant vapor provide a method by which one may perform a sterilization procedure on intricate devices at low temperatures under reduced pressures in shorter time periods. A liquid solution that contains an MME may advantageously be used at room temperature under reduced pressure due to the significantly higher vapor pressure of the MME over that of its counterpart peroxy acid.

Under reduced pressure, the MME will readily self-vaporize and subsequently initiate vaporization of the peroxy acid liquid after which point the MME vapor molecules “transport” peroxy acid vapor molecules evenly throughout chamber. This phenomenon increases flow of the sterilant vapor to areas of restricted flow such as small crevices or narrow openings or lumens of devices. The peri-peroxy acid compounds (e.g., sterilants) and, if present, MME agents may be utilized within a sterilization system including a vacuum drying chamber. The drying chamber (also referred to as a sterilization chamber herein) can create a conductively heated, negative pressure (e.g., vacuum) environment, which can be used to activate and/or otherwise promote the sterilization of devices, including electronic devices, to at least a desired threshold sterilization level (e.g., sterility assurance level, or SAL).

In some cases, the devices to be sterilized are medical devices, such as medical implants, instruments, and/or other devices; and the sterilization is to a level acceptable for the medical context of the device. In one embodiment, a medical device (e.g., an endoscope) that has been exposed to excessive contamination is placed inside a sterilization chamber. The sterilizing chamber is closed and a sterilizing routine commences. During the sterilizing routine, the chamber is pressurized to a vacuum level sufficient to gasify liquids inside or on a device, and the device is conductively heated at least to replace latent heat of vaporization lost during the pressurization.

In an arrangement, the negative pressure and/or heat can cause a sterilant present in the sterilization chamber to be activated. For example, sterilant is provided in the form of a matrix that will off-gas under vacuum and heat, which can increase the efficacy of the system and can reduce the time for complete sterilization (e.g., to the desired SAL). In a further arrangement, the sterilant may be introduced into the chamber via, for example, an ampoule or cartridge system. Further, the off-gassing or other introduction of the sterilant in a vacuum environment permits expansion (e.g., adiabatic expansion) of the gasified sterilant, which may readily enter into evacuated internal regions of the device.

In embodiments, the MME-sterilant composition, such as solid forms described herein, is placed, manually or automatically, into the process chamber of the vacuum system after which point the door(s) to the system are closed and sealed and the system is pressurized by the pressurizing subsystem. The heating subsystem optionally provides heat to the conductive thermal assembly of the process chamber. As the pressure in the chamber is reduced the MME-transport moiety vapor from the solid is outgassed. In an embodiment, the pressure in the process chamber is reduced to below 1 torr. In an embodiment, the pressure in the process chamber is reduced to below 0.1 torr. In an embodiment, the pressure in the process chamber is reduced to 1×10-3 torr or less. Heated or non-heated, non-reactive carrier gas may be introduced into the process chamber enhancing flow of the MME-transport moiety vapor throughout the process chamber.

### Peri-Peroxy Solution Stabilization Matrix

In another embodiment, peri-peroxy acid is vaporized, preferably, from a liquid containing a peri-peroxy acid solution that may contain one or more peroxy acids and one or more MMEs that are encapsulated in a stabilizing matrix. The stabilizing matrix that contains the peri-peroxy acid is called the sterilant matrix and is ultimately placed in the sterilizing system (e.g., sterilization chamber), to undergo the sterilizing routine in which the chamber is pressurized to a vacuum level sufficient to gasify the peri-peroxy acid solution within the sterilant matrix. The peri-peroxy acid solution can be stabilized in a material matrix. It is in this material matrix that peri-peroxy acid solution molecules are reversibly trapped in the interior of the matrix to prevent reaction and to suppress degradation. The stabilization matrix may consist of synthetic polymers, biopolymers, ceramics, glass, or composite materials, or any combination of these materials. Some examples of synthetic polymers that may be used include, but are not limited to, silicones, polyacrylates, polyethylenes and related polymers, polyamides, polyurethanes, polyethers, polyphosphazenes, polyanhydrides, polyacetals, poly(ortho esters), polyphosphoesters, polycaprolactones, polylactides, and polycarbonates. Some examples of biopolymers that may be used include, but are not limited to, carbohydrates, starches, celluloses, chitosans, chitins, dextrans, gelatins, lignins and polyamino acids. Some examples of ceramics include, but are not limited to, aluminum oxide, zirconium oxide, silicon dioxide, magnesium oxide, titanium oxide, aluminum nitride, silicon nitride, boron nitride and silicon carbide. Composite materials may consist, but are not limited to, two or more of the matrix materials listed above.

The peri-peroxy acid solution may be prepared in liquid form using any of the methods described previously in this disclosure and then immediately mixed or loaded into the stabilization matrix. The sterilant matrix is used to form a gel or a solid that contains the peri-peroxy acid molecules. The sterilant gel or solid may be shaped into the form of beads, a block and/or may be stored in a container or wrapping that is sufficiently porous and such that allows the peroxy acid vapor molecules to outgas from the material matrix and/or container when exposed to reduced pressure and/or increased temperature. The purity and concentration of peroxy acid loaded into the material matrix can be adjusted according to the intended use. It may be seen that a sterilant gel or solid that contains a purer and more concentrated form of peroxy acid may be used for multiple sterilization runs, for killing more resistant microbes, and/or for sterilizing a larger bioload. The sterilizing system can be configured for a user to easily and safely place the sterilant gel or solid into the chamber before pressurizing the system and prior to initiating a sterilization cycle. This embodiment also provides a simplified method for extending the shelf-life of the peri-peroxy acid in such a way that the sterilant does not need to be prepared just prior to use. It may sit at a reasonable temperature without spontaneously degrading for an extended period giving the user the opportunity to pull the sterilant gel or solid off of the self when needed to insert it into the sterilizing system for one or more uses. When contained in a stabilization matrix, peroxy acid can be safely produced in bulk for use within sterilizing systems, such as those described herein.

### Packaging and Delivery Device

### Ampoule System

In another embodiment, the component of the sterilant, e.g., peri-peroxy acid, component chemicals, carboxylic acid and/or carboxylic acid source, hydrogen peroxide and/or reactive oxygen species, are packaged separately as part of a multi-component ampoule system and is used for preparing an active peri-peroxy acid liquid solution just prior to use in a sterilizing vaporization system. In one aspect of the embodiment, the component chemicals can first be mixed inside the ampoule or ampoule housing and then second, can be injected or dispersed into a chemical mixing apparatus of the sterilizing system, or directly into the sterilizing system itself. In a second aspect of this embodiment, the component chemicals can be mixed outside of the ampoule or ampoule housing after the isolated chemicals are injected or dispersed into a chemical mixing apparatus of the sterilizing system, or directly into the sterilizing system itself. The chemical mixing apparatus or the sterilizing system is configured to receive a multi-component ampoule in such a way that the ampoule, at the proximal end, is locked into a port of the system. The proximal end of the ampoule that is locked into the system contains a membrane that is capable of being pierced by a needle or a cannula and a connector, such as a luer lock, that locks the ampoule housing into the system. The pierceable membrane of the ampoule seals off the contents of the ampoule from the system until pierced by a needle or other such device of the chemical mixing apparatus or the sterilizing system. Once pierced, the mixed or isolated chemicals of the ampoule are injected or dispersed into the system for further mixing, heating, and vaporization. Located distally from the lock assembly and the membrane is the ampoule housing that may be in the shape of a cylinder. The ampoule housing is made of a thermoplastic known in the art. Within the ampoule housing is a compartment that may be in the shape of a tube. This tube is made of glass that extends internally into the ampoule housing from the distal end of the ampoule. There may be one or more compartments per ampoule housing. Each compartment is a separate storage unit and is designed to hold a volume of liquid or solid chemical. The ampoule housing is also a separate storage unit designed to hold a volume of liquid or solid chemical. The volume of chemical that each compartment can hold can be altered by increasing or decreasing the size of the compartment and/or the size of the ampoule housing. The compartment is attached to a flange at the distal end of the ampoule housing and is sealed by a plug. The separation of ampoule housing from the compartment or compartments are meant to keep the chemicals stored until need by the user.

### Cartridge System

Another embodiment may contain separate cartridges that are used to store the component chemicals of the sterilant solution, e,g., peri-peroxy acid solution. Preferably, the sterilant is peri-peroxy acid, and the component chemicals, one or more carboxylic acid and/or carboxylic acid source, and hydrogen peroxide and/or a reactive oxygen species are stored separately in individual cartridges. The cartridges are designed to hold a volume of liquid and may be sealed under vacuum. The cartridges are designed to plug into the side of the chemical mixing apparatus of the sterilization system or directly into the side of the sterilization system itself The sterilization system is configured to receive these cartridges in such a way that the cartridges, at the proximal end, are locked into the system. The proximal end of the cartridge that is locked into the system contains membrane that is capable of being pierced. The pierceable membrane of the cartridge seals off the contents of the cartridge from the system until pierced by a needle or cannula of the chemical mixing apparatus or the sterilization system. Chemicals stored in each of the cartridges are injected or dispersed into the chemical mixing apparatus or the sterilization system where the chemical components are mixed, placed under reduced pressure and/or heated to form performic acid vapor. The sterilization system can be programmed to withdraw any volume of chemical from each cartridge to generate the desired amount and concentration of performic acid sterilant. The separation of component chemicals in cartridges are meant to safely keep the chemicals stored until just prior to use. Also, this embodiment provides a method for safely generating a peracid liquid vapor source at higher concentrations for shorter sterilization run times, if need be, as mixing of the component chemicals followed by controlled heating and vaporization of the sterilant will be performed under reduced pressure in a vacuum system.

While the materials, methods and apparatus of the disclosure have been particularly shown and described with reference to the apparatus and methods herein, it will be understood by those skilled in the art that various changes in the form and details may be made therein without departing from the spirit and scope of the invention. While the method has been specifically described for treatment of a lumen, it will be readily apparent to one of ordinary skill in the art that a variety of articles including substrates, devices including medical devices, and electronic devices (e.g., computers) can be treated using the methods as described herein.

When a Markush group or other grouping is used herein, all individual members of the group and all combinations and subcombinations possible of the group are intended to be individually included in the disclosure. Every formulation or combination of components described or exemplified can be used to practice the invention, unless otherwise stated. Specific names of compounds are intended to be exemplary, as it is known that one of ordinary skill in the art can name the same compounds differently.

One of ordinary skill in the art will appreciate that methods, including experimental procedure, preparation methods and analytical methods, materials and device elements other than those specifically exemplified can be employed in the practice of the invention without resort to undue experimentation. All art-known functional equivalents of any such methods or materials are intended to be included in this invention.

Whenever a range is given in the specification, for example, a composition range, a range of process conditions, a range of pressures or temperatures or the like, all intermediate ranges and subranges, as well as all individual values included in the ranges given are intended to be included in the disclosure.

The invention illustratively described herein suitably may be practiced in the absence of any element or elements, limitation or limitations which is not specifically disclosed herein.

Without wishing to be bound by any particular theory, there can be discussion herein of beliefs or understandings of underlying principles or mechanisms of action relating to the invention. It is recognized that regardless of the ultimate correctness of any mechanistic explanation or hypothesis, an embodiment of the invention can nonetheless be operative and useful.

All references throughout this application, for example patent documents including issued or granted patents or equivalents; patent application publications; and non-patent literature documents or other source material; are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this disclosure.

### THE EXAMPLES

### Example 1—Method for Preparing Peri-Peroxy Acid Sterilant Solutions from Carboxylic Acids, Hydrogen Peroxide and MME's

Peri-peroxy acid solutions (35 g) were generated by mixing a carboxylic acid (Acetic Acid, 98% (Sigma Aldrich, St. Louis, Mo.), Formic Acid, 98% (Sigma Aldrich, St. Louis, Mo.) or Propionic Acid, 99.5% (Sigma Aldrich, St. Louis, Mo.)), 30 wt % aqueous hydrogen peroxide (Sigma Aldrich, St. Louis, Mo.) and an MME (Diethyl Ether, 99.7% (Sigma Aldrich, St. Louis, Mo.), Methanol, 99.9% (Sigma Aldrich, St. Louis, Mo.) and Methyl Methanoate, 99% (Sigma Aldrich, St, Louis, Mo.) according to the embodiments described above. Reaction contents are shown in Table 1. The sterilant solutions are then transferred to a container or wrapping sufficiently porous to emit vapors.

### Example 2—Sterilization of a Surgical Scalpel, a Fabric, and a Foley Catheter Using Peri-Peroxy Acid Sterilant Solutions Generated from Carboxylic Acids, Hydrogen Peroxide and MME's

Peri-peroxy acid solutions (35 g) from Example 1, a scalpel, a piece of fabric, a foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to a base pressure of 200 mTorr while heat of 40° C. is evenly applied to the chamber. Once the base pressure is reached, the system is allowed to remain in a steady state of about 1 Torr for 15 minutes. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test are seen when the color of the media does not change color. This result indicates the peri-peroxy acid sterilant solution under the descried sterilization chamber run conditions was able to successfully permeate through the porous transport filter of the biological indicator and achieve an SAL of 1×10E-6. Negative (FAIL) results from a biological indicator test are seem when the color of the media does change color, indicating an SAL of 1×10-6 was not achieved under the described sterilization process conditions. Results from biological indicator tests are listed in Table 1.

### Example 3—Method for Preparing Peri-Peroxy Acid Sterilization Solutions from Alternative Carboxylic Acid Sources, a Reactive Oxygen Species and MME's

Peri-peroxy acid solutions (35 g) were generated my mixing an alternative source of carboxylic acid (Sodium Acetate, 99% (Sigma Aldrich, St. Louis, Mo.), Sodium Formate, 99% (Sigma Aldrich, St. Louis, Mo.) or Sodium Propionate (Sigma Aldrich, St. Louis, Mo.)), 30 wt % aqueous hydrogen peroxide (Sigma Aldrich, St. Louis, Mo.) and an MME (Diethyl Ether, 99.7% (Sigma Aldrich, St. Louis, Mo.), Methanol, 99.9% (Sigma Aldrich, St. Louis, Mo.) or Methyl Methanoate, 99% (Sigma Aldrich, St. Louis, Mo.) according to the embodiments described above. Reaction contents are shown in Table 2. The sterilant solutions are then transferred to a container or wrapping sufficiently porous to emit vapors.

TABLE 1

| | | Carboxylic | Hydrogen | | | |
|---|---|---|---|---|---|---|
| | Carboxylic | Acid | Peroxide | | MME | B.I. Test |
| Formulation | Acid | (wt %) | (aq) (wt %) | MME | (wt %) | Result |
| 1 | Acetic acid | 40 | 45 | Diethyl ether | 15 | PASS |
| 2 | Acetic acid | 50 | 45 | Diethyl ether | 5 | PASS |
| 3 | Acetic acid | 50 | 50 | Diethyl ether | 0 | PASS |
| 4 | Acetic acid | 40 | 45 | Methanol | 15 | PASS |
| 5 | Acetic acid | 50 | 45 | Methanol | 5 | PASS |
| 6 | Acetic acid | 50 | 50 | Methanol | 0 | PASS |
| 7 | Acetic acid | 40 | 45 | Methyl Methanoate | 15 | PASS |
| 8 | Acetic acid | 50 | 45 | Methyl Methanoate | 5 | PASS |
| 9 | Acetic acid | 50 | 50 | Methyl Methanoate | 0 | PASS |
| 10 | Formic acid | 40 | 45 | Diethyl ether | 15 | PASS |
| 11 | Formic acid | 50 | 45 | Diethyl ether | 5 | PASS |
| 12 | Formic acid | 50 | 50 | Diethyl ether | 0 | PASS |
| 13 | Formic acid | 40 | 45 | Methanol | 15 | PASS |
| 14 | Formic acid | 50 | 45 | Methanol | 5 | PASS |
| 15 | Formic acid | 50 | 45 | Methanol | 0 | PASS |
| 16 | Formic acid | 40 | 45 | Methyl Methanoate | 15 | PASS |
| 17 | Formic acid | 50 | 45 | Methyl Methanoate | 5 | PASS |
| 18 | Formic acid | 50 | 50 | Methyl Methanoate | 0 | PASS |
| 19 | Propionic acid | 40 | 45 | Diethyl ether | 15 | PASS |
| 20 | Propionic acid | 50 | 45 | Diethyl ether | 5 | PASS |
| 21 | Propionic acid | 50 | 50 | Diethyl ether | 0 | PASS |
| 22 | Propionic acid | 40 | 45 | Methanol | 15 | PASS |
| 23 | Propionic acid | 50 | 45 | Methanol | 5 | PASS |
| 24 | Propionic acid | 50 | 50 | Methanol | 0 | PASS |
| 25 | Propionic acid | 40 | 45 | Methyl Methanoate | 15 | PASS |
| 26 | Propionic acid | 50 | 45 | Methyl Methanoate | 5 | PASS |
| 27 | Propionic acid | 50 | 50 | Methyl Methanoate | 0 | PASS |
| 28 | Acetic acd | 25 | 40 | Diethyl ether | 10 | PASS |
| | Formic acid | 25 | | | | |
| 29 | Acetic acid | 25 | 40 | Methanol | 10 | PASS |
| | Formic acid | 25 | | | | |
| 30 | Acetic acid | 25 | 40 | Methyl Methanoate | 10 | PASS |
| | Formic acid | 25 | | | | |

### Example 4—Sterilization of a Surgical Scalpel, a Fabric, and a Foley Catheter Using Peri-Peroxy Acid Sterilant Solutions Generated from Alternative Carboxylic Acid Sources, a Reactive Oxygen Species and MME's

Peri-peroxy acid solutions (35 g) from Example 3, a scalpel, a piece of fabric, a foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to a base pressure of 200 mTorr while heat of 40° C. is evenly applied to the chamber. Once the base pressure is reached, the system is allowed to remain in a steady state of about 1 Torr for 15 minutes. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test are seen when the color of the media does not change color. This result indicates the peri-peroxy acid sterilant solution under the descried sterilization chamber run conditions was able to successfully permeate through the porous transport filter of the biological indicator and achieve an SAL of 1×10E-6. Negative (FAIL) results from a biological indicator test are seem when the color of the culture media does change color, indicating an SAL of 1×10-6 was not achieved under the described sterilization process conditions. Results from biological indicator tests are listed in Table 2.

### Example 5—Method for Preparing Peri-Peroxy Acid Sterilization Solutions from Carboxylic Acids, Hydrogen Peroxide, MME's and a Catalyst

Peri-peroxy acid solutions (35 g) were generated my mixing a carboxylic acid (Acetic Acid, 98% (Sigma Aldrich, St. Louis, Mo.), Formic Acid, 98% (Sigma Aldrich, St. Louis, Mo.), Propionic Acid, 99.5% (Sigma Aldrich, St. Louis, Mo.), aqueous hydrogen peroxide, 30 wt % (Sigma Aldrich, St. Louis, Mo.), MME (Diethyl Ether, 99.7% (St. Louis, Mo.) and a catalyst (Sulfuric Acid, 99.9999% (Sigma Aldrich, St. Louis, Mo.) according to the embodiments described above. Reaction contents are shown in Table 3. The sterilant solutions are then transferred to a container or wrapping sufficiently porous to emit vapors.

TABLE 2

| | Alternative | Alternative | Hydrogen | | | |
|---|---|---|---|---|---|---|
| | Carboxylic | Carboxylic Acid | Peroxide | | MME | B.I. Test |
| Formulation | Acid Source | Source (wt %) | (aq) (wt %) | MME | (wt %) | Result |
| 1 | Sodium Acetate | 60 | 25 | Diethyl ether | 15 | PASS |
| 2 | Sodium Acetate | 50 | 45 | Diethyl ether | 5 | PASS |
| 3 | Sodium Acetate | 50 | 50 | Diethyl ether | 0 | PASS |
| 4 | Sodium Acetate | 60 | 25 | Methanol | 15 | PASS |
| 5 | Sodium Acetate | 50 | 45 | Methanol | 5 | PASS |
| 6 | Sodium Acetate | 50 | 50 | Methanol | 0 | PASS |
| 7 | Sodium Acetate | 60 | 25 | Methyl Methanoate | 15 | PASS |
| 8 | Sodium Acetate | 50 | 45 | Methyl Methanoate | 5 | PASS |
| 9 | Sodium Acetate | 50 | 50 | Methyl Methanoate | 0 | PASS |
| 10 | Sodium Formate | 60 | 25 | Diethyl ether | 15 | PASS |
| 11 | Sodium Formate | 50 | 45 | Diethyl ether | 5 | PASS |
| 12 | Sodium Formate | 50 | 50 | Diethyl ether | 0 | PASS |
| 13 | Sodium Formate | 60 | 25 | Methanol | 15 | PASS |
| 14 | Sodium Formate | 50 | 45 | Methanol | 5 | PASS |
| 15 | Sodium Formate | 50 | 50 | Methanol | 0 | PASS |
| 16 | Sodium Formate | 60 | 25 | Methyl Methanoate | 15 | PASS |
| 17 | Sodium Formate | 50 | 45 | Methyl Methanoate | 5 | PASS |
| 18 | Sodium Formate | 50 | 50 | Methyl Methanoate | 0 | PASS |
| 19 | Sodium Propionate | 60 | 25 | Diethyl ether | 15 | PASS |
| 20 | Sodium Propionate | 50 | 45 | Diethyl ether | 5 | PASS |
| 21 | Sodium Propionate | 50 | 50 | Diethyl ether | 0 | PASS |
| 22 | Sodium Propionate | 60 | 25 | Methanol | 15 | PASS |
| 23 | Sodium Propionate | 50 | 45 | Methanol | 5 | PASS |
| 24 | Sodium Propionate | 50 | 50 | Methanol | 0 | PASS |
| 25 | Sodium Propionate | 60 | 25 | Methyl Methanoate | 15 | PASS |
| 26 | Sodium Propionate | 50 | 45 | Methyl Methanoate | 5 | PASS |
| 27 | Sodium Propionate | 50 | 50 | Methyl Methanoate | 0 | FAIL |
| 28 | Sodium Acetate | 30 | 30 | Diethyl ether | 10 | PASS |
| | Sodium Formate | 30 | | | | |
| 29 | Sodium Acetate | 30 | 30 | Methanol | 10 | PASS |
| | Sodium Formate | 30 | | | | |
| 30 | Sodium Acetate | 30 | 30 | Methyl Methanoate | 10 | PASS |
| | Sodium Formate | 30 | | | | |

### Example 6—Sterilization of a Surgical Scalpel, a Fabric, and a Foley Catheter Using Peri-Peroxy Acid Sterilant Solutions Generated from Carboxylic Acids, a Hydrogen Peroxide, MME's and a Catalyst

Peri-peroxy acid solutions (35 g) from Example 5, a scalpel, a piece of fabric, a foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to a base pressure of 200 mTorr while heat of 40° C. is evenly applied to the chamber. Once the base pressure is reached, the system is allowed to remain in a steady state of about 1 Torr for 15 minutes. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test are seen when the color of the media does not change color. This result indicates the peri-peroxy acid sterilant solution under the desired sterilization chamber run conditions was able to successfully permeate through the porous transport filter of the biological indicator and achieve an SAL of 1×10E-6. Negative (FAIL) results from the biological indicator test are seen when the color of the culture media does change color, indicating an SAL of 1×10-6 was not achieved under the described sterilization process conditions. Results from biological indicator tests are listed in Table 5.

TABLE 3

| | Carboxylic | Carboxylic | Hydrogen Peroxide | | MME | | Catalyst | B.I. Test |
|---|---|---|---|---|---|---|---|---|
| Formulation | Acid | Acid (wt %) | (aq) (wt %) | MME | (wt %) | Catalyst | (wt %) | Result |
| 1 | Acetic acid | 50 | 34.5 | Diethyl ether | 15 | Sulfuric Acid | 0.5 | PASS |
| 2 | Acetic acid | 40 | 54.5 | Diethyl ether | 5 | Sulfuric Acid | 0.5 | PASS |
| 3 | Acetic acid | 50 | 49.5 | Diethyl ether | 0 | Sulfuric Acid | 0.5 | PASS |
| 4 | Formic acid | 50 | 34.5 | Diethyl ether | 15 | Sulfuric Acid | 0.5 | PASS |
| 5 | Formic acid | 40 | 54.5 | Diethyl ether | 5 | Sulfuric Acid | 0.5 | PASS |
| 6 | Formic acid | 50 | 49.5 | Diethyl ether | 0 | Sulfuric Acid | 0.5 | PASS |
| 7 | Propionic acid | 50 | 34.5 | Diethyl ether | 15 | Sulfuric Acid | 0.5 | PASS |
| 8 | Propionic acid | 40 | 54.5 | Diethyl ether | 5 | Sulfuric Acid | 0.5 | PASS |
| 9 | Propionic acid | 50 | 49.5 | Diethyl ether | 0 | Sulfuric Acid | 0.5 | PASS |
| 10 | Acetic acd | 25 | 39.5 | Diethyl ether | 10 | Sulfuric Acid | 0.5 | PASS |
| | Formic acid | 25 | | | | | | |

### Example 7—Method for Preparing Peri-Peroxy Acid Sterilization Solution from Carboxylic Acids. Hydrogen Peroxide, MME and a Stabilizer

Peri-peroxy acid solutions (35 g) were generated my mixing a carboxylic acid (Acetic Acid, 98% (Sigma Aldrich, St. Louis Mo.), Formic Acid, 98% (Sigma Aldrich, St. Louis Mo.) or Propionic Acid, 99.5% (Sigma Aldrich, St. Louis Mo.), aqueous hydrogen peroxide, 30 wt % (Sigma Aldrich, St. Louis Mo.), MME (Diethyl Ether, 99.7% (Sigma Aldrich, St. Louis Mo.) and a stabilizer (Dipicolinic Acid, 99% (Sigma Aldrich, St. Louis Mo.) according to the embodiments described above. Reaction contents are shown in Table 4. The sterilant solutions are then transferred to a container or wrapping sufficiently porous to emit vapors.

### Example 8—Sterilization of a Surgical Scalpel, a Fabric, and a Foley Catheter Using Peri-Peroxy Acid Sterilant Solutions Generated from Carboxylic Acids, a Hydrogen Peroxide, MME and a Stabilizer

Peri-peroxy acid solutions (35 g) from Example 7, a scalpel, a piece of fabric, a foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to a base pressure of 200 mTorr while heat of 40° C. is evenly applied to the chamber. Once the base pressure is reached, the system is allowed to remain in a steady state of about 1 Torr for 15 minutes. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test are seen when the color of the media does not change color. This result indicates the peri-peroxy acid sterilant solution under the desired sterilization chamber run conditions was able to successfully achieve an SAL of 1×10E-6. Negative (FAIL) results from a biological indicator test are seen when the color of the culture media does change color indicating an SAL of 1×10-6 was not achieved under the described sterilization process conditions. Results from biological indicator tests are listed in Table 4.

TABLE 4

| | Carboxylic | Carboxylic | Hydrogen Peroxide | | MME | | Stabilizer | B.I. Test |
|---|---|---|---|---|---|---|---|---|
| Formulation | Acid | Acid (wt %) | (aq) (wt %) | MME | (wt %) | Stabilizer | (wt %) | Result |
| 1 | Acetic acid | 50 | 31 | Diethyl ether | 15 | Dipicolinic Acid | 4 | PASS |
| 2 | Acetic acid | 40 | 51 | Diethyl ether | 5 | Dipicolinic Acid | 4 | PASS |
| 3 | Acetic acid | 50 | 46 | Diethyl ether | 0 | Dipicolinic Acid | 4 | PASS |
| 4 | Formic acid | 50 | 31 | Diethyl ether | 15 | Dipicolinic Acid | 4 | PASS |
| 5 | Formic acid | 40 | 51 | Diethyl ether | 5 | Dipicolinic Acid | 4 | PASS |
| 6 | Formic acid | 50 | 46 | Diethyl ether | 0 | Dipicolinic Acid | 4 | PASS |
| 7 | Propionic acid | 50 | 31 | Diethyl ether | 15 | Dipicolinic Acid | 4 | PASS |
| 8 | Propionic acid | 40 | 51 | Diethyl ether | 5 | Dipicolinic Acid | 4 | PASS |
| 9 | Propionic acid | 50 | 46 | Diethyl ether | 0 | Dipicolinic Acid | 4 | PASS |
| 10 | Acetic acd | 25 | 36 | Diethyl ether | 10 | Dipicolinic Acid | 4 | PASS |
| | Formic acid | 25 | | | | | | |

### Example 9—Method for Preparing Peri-Peroxy Acid Sterilization Solutions from Carboxylic Acids, Hydrogen Peroxide, MME, a Catalyst and a Stabilizer

Peri-peroxy acid solutions (35 g) were generated my mixing a carboxylic acid (Acetic Acid, 98% (Sigma Aldrich, St. Louis, Mo.), Formic Acid, 98% (Sigma Aldrich, St. Louis, Mo.) or Propionic Acid, 99.5% (Sigma Aldrich, St. Louis, Mo.)), aqueous hydrogen peroxide, 30 wt % (Sigma Aldrich, St. Louis, Mo.), MME (Diethyl Ether, 99.7% (Sigma Aldrich, St. Louis, Mo.)), a catalyst (Sulfuric Acid, 99.9999% (Sigma Aldrich, St. Louis, Mo.)) and a stabilizer (Dipicolinic Acid, 99% (Sigma Aldrich, St. Louis, Mo.) according to the embodiments described above. Reaction contents are shown in Table 5. The sterilant solutions are then transferred to a container or wrapping sufficiently porous to emit vapors.

### Example 10—Sterilization of a Surgical Scalpel, a Fabric, and a Foley Catheter Using Peri-Peroxy Acid Sterilant Solutions Generated from Carboxylic Acids, a Hydrogen Peroxide, MME Catalyst and a Stabilizer

Peri-peroxy acid solutions (35 g) from Example 9, a scalpel, a piece of fabric, a foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to a base pressure of 200 mTorr while heat of 40° C. is evenly applied to the chamber. Once the base pressure is reached, the system is allowed to remain in a steady state of about 1 Torr for 15 minutes. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test are seen when the color of the media does not change color. This result indicates the peri-peroxy acid sterilant solution under the descried sterilization chamber run conditions was able to successfully permeate through the porous transport filter of the biological indicator and achieve an SAL of 1×10E-6. Negative (FAIL) results from a biological indicator test are seen when the color of the media does change color, indicating an SAL of 1×10-6 was not achieved under the described sterilization process conditions. Results from biological indicator tests are listed in Table 5.

TABLE 5

| | | Carboxylic | Hydrogen | | | | | | | B.I. |
|---|---|---|---|---|---|---|---|---|---|---|
| | Carboxylic | Acid | Peroxide | | MME | | Catalyst | | Stabilizer | Test |
| Formulation | Acid | (wt %) | (aq) (wt %) | MME | (wt %) | Catalyst | (wt %) | Stabilizer | (wt %) | Result |
| 1 | Acetic acid | 50 | 30.5 | Diethyl ether | 15 | Sulfuric Acid | 0.5 | Dipicolinic Acid | 4 | PASS |
| 2 | Acetic acid | 40 | 50.5 | Diethyl ether | 5 | Sulfuric Acid | 0.5 | Dipicolinic Acid | 4 | PASS |
| 3 | Acetic acid | 50 | 45.5 | Diethyl ether | 0 | Sulfuric Acid | 0.5 | Dipicolinic Acid | 4 | PASS |
| 4 | Formic acid | 50 | 30.5 | Diethyl ether | 15 | Sulfuric Acid | 0.5 | Dipicolinic Acid | 4 | PASS |
| 5 | Formic acid | 40 | 50.5 | Diethyl ether | 5 | Sulfuric Acid | 0.5 | Dipicolinic Acid | 4 | PASS |
| 6 | Formic acid | 50 | 45.5 | Diethyl ether | 0 | Sulfuric Acid | 0.5 | Dipicolinic Acid | 4 | PASS |
| 7 | Propionic acid | 50 | 30.5 | Diethyl ether | 15 | Sulfuric Acid | 0.5 | Dipicolinic Acid | 4 | PASS |
| 8 | Propionic acid | 40 | 50.5 | Diethyl ether | 5 | Sulfuric Acid | 0.5 | Dipicolinic Acid | 4 | PASS |
| 9 | Propionic acid | 50 | 45.5 | Diethyl ether | 0 | Sulfuric Acid | 0.5 | Dipicolinic Acid | 4 | PASS |
| 10 | Acetic acd | 25 | 35.5 | Diethyl ether | 10 | Sulfuric Acid | 0.5 | Dipicolinic Acid | 4 | PASS |
| | Formic acid | 25 | | | | | | | | |

### Example 11—Method for Preparing Peri-Peroxy Acid Sterilization Solution from Alternative Carboxylic Acid Sources, a Reactive Oxygen Species, an MME and a Catalyst

Peri-peroxy acid solutions (35 g) were generated my mixing an alternative carboxylic acid source (Sodium Acetate, 99% (Sigma Aldrich, St. Louis, Mo.), Sodium Formate, 99% (Sigma Aldrich, St. Louis, Mo.) or Sodium Propionate (Sigma Aldrich, St. Louis, Mo.)), aqueous hydrogen peroxide, 30 wt % (Sigma Aldrich, St. Louis, Mo.), an MME (Diethyl Ether, 99.7% (Sigma Aldrich, St. Louis, Mo.) and a catalyst (Sulfuric Acid, 99.9999% (Sigma Aldrich, St. Louis, Mo.) according to the embodiments described above. Reaction contents are shown in Table 6. The sterilant solutions are then transferred to a container or wrapping sufficiently porous to emit vapors.

### Example 12—Sterilization of a Surgical Scalpel, a Fabric, and a Foley Catheter Using Peri-Peroxy Acid Sterilant Solutions Generated from Carboxylic Acids, a Hydrogen Peroxide, an MME and a Catalyst

Peri-peroxy acid solutions (35 g) from Example 11, a scalpel, a piece of fabric, a foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to a base pressure of 200 mTorr while heat of 40° C. is evenly applied to the chamber. Once the base pressure is reached, the system is allowed to remain in a steady state of about 1 Torr for 15 minutes. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58°□C. after which time the color of the media is visualized for color change. Positive (PASS) results from a biological indicator test are seen when the color of the media does not change color. This result indicates the peri-peroxy acid sterilant solution under the descried sterilization chamber run conditions was able to successfully permeate through the porous transport filter of the biological indicator and achieve an SAL of 1×10E-6. Negative (FAIL) results from a biological indicator test are seen when the color of the media does change color, indicating an SAL of 1×10-6 was not achieved under the described sterilization process conditions. Results from biological indicator tests are listed in Table 6.

TABLE 6

| | Alternative Carboxylic | Alternative Carboxylic | Hydrogen Peroxide | | MME | | Catalyst | B.I. Test |
|---|---|---|---|---|---|---|---|---|
| Formulation | Acid Source | Acid Source (wt %) | (aq) (wt %) | MME | (wt %) | Catalyst | (wt %) | Result |
| 1 | Sodium Acetate | 50 | 34.5 | Diethyl ether | 15 | Sulfuric Acid | 0.5 | PASS |
| 2 | Sodium Acetate | 40 | 54.5 | Diethyl ether | 5 | Sulfuric Acid | 0.5 | PASS |
| 3 | Sodium Acetate | 50 | 49.5 | Diethyl ether | 0 | Sulfuric Acid | 0.5 | PASS |
| 4 | Sodium Formate | 50 | 34.5 | Diethyl ether | 15 | Sulfuric Acid | 0.5 | PASS |
| 5 | Sodium Formate | 40 | 54.5 | Diethyl ether | 5 | Sulfuric Acid | 0.5 | PASS |
| 6 | Sodium Formate | 50 | 49.5 | Diethyl ether | 0 | Sulfuric Acid | 0.5 | PASS |
| 7 | Sodium Propionate | 50 | 34.5 | Diethyl ether | 15 | Sulfuric Acid | 0.5 | PASS |
| 8 | Sodium Propionate | 40 | 54.5 | Diethyl ether | 5 | Sulfuric Acid | 0.5 | PASS |
| 9 | Sodium Propionate | 50 | 49.5 | Diethyl ether | 0 | Sulfuric Acid | 0.5 | PASS |
| 10 | Sodium Acetate | 25 | 39.5 | Diethyl ether | 10 | Sulfuric Acid | 0.5 | PASS |
| | Sodium Formate | 25 | | | | | | |

### Example 13—Method for Preparing Peri-Peroxy Acid Sterilization Solutions from Alternative Carboxylic Acid Sources, a Reactive Oxygen Species, an MME and a Stabilizer

Peri-peroxy acid solutions (35 g) were generated my mixing an alternative carboxylic acid source (Sodium Acetate, 99% (Sigma Aldrich, St. Louis, Mo.), Sodium Formate, 99% (Sigma Aldrich, St. Louis, Mo.) or Sodium Propionate (Sigma Aldrich, St. Louis, Mo.)), aqueous hydrogen peroxide, 30 wt % (Sigma Aldrich, St. Louis, Mo.) an MME (Diethyl Ether, 99.7% (Sigma Aldrich, St. Louis, Mo.)) and a stabilizer (Dipicolinic Acid, 99% (Sigma Aldrich, St. Louis, Mo.)) according to the embodiments described above. Reaction contents are shown in Table 7. The sterilant solutions are then transferred to a container or wrapping sufficiently porous to emit vapors.

### Example 14—Sterilization of a Surgical Scalpel, a Fabric, and a Foley Catheter Using Peri-Peroxy Acid Sterilant Solutions Generated from an Alternative Carboxylic Acid Source, a Reactive Oxygen Species, an MME and a Stabilizer

Peri-peroxy acid solutions (35 g) from Example 13, a scalpel, a piece of fabric, a foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to a base pressure of 200 mTorr while heat of 40° C. is evenly applied to the chamber. Once the base pressure is reached, the system is allowed to remain in a steady state of about 1 Torr for 15 minutes. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58°□C. after which time the color of the media is visualized for color change. Positive (PASS) results from a biological indicator test are seen when the color of the media does not change color. This result indicates the peri-peroxy acid sterilant solution under the descried sterilization chamber run conditions was able to successfully permeate through the porous transport filter of the biological indicator and achieve an SAL of 1×10E-6. Negative (FAIL) results from a biological indicator test are seen when the color of the media does change color, indicating an SAL of 1×10-6 was not achieved under the described sterilization process conditions. Results from biological indicator tests are listed in Table 7.

TABLE 7

| | Alternative Carboxylic | Alternative Carboxylic | Hydrogen Peroxide | | MME | | Stabilizer | B.I. Test |
|---|---|---|---|---|---|---|---|---|
| Formulation | Acid Source | Acid Source (wt %) | (aq) (wt %) | MME | (wt %) | Stabilizer | (wt %) | Result |
| 1 | Sodium Acetate | 50 | 31 | Diethyl ether | 15 | Dipicolinic Acid | 4 | PASS |
| 2 | Sodium Acetate | 40 | 51 | Diethyl ether | 5 | Dipicolinic Acid | 4 | PASS |
| 3 | Sodium Acetate | 50 | 46 | Diethyl ether | 0 | Dipicolinic Acid | 4 | PASS |
| 4 | Sodium Formate | 50 | 31 | Diethyl ether | 15 | Dipicolinic Acid | 4 | PASS |
| 5 | Sodium Formate | 40 | 51 | Diethyl ether | 5 | Dipicolinic Acid | 4 | PASS |
| 6 | Sodium Formate | 50 | 46 | Diethyl ether | 0 | Dipicolinic Acid | 4 | PASS |
| 7 | Sodium Propionate | 50 | 31 | Diethyl ether | 15 | Dipicolinic Acid | 4 | PASS |
| 8 | Sodium Propionate | 40 | 51 | Diethyl ether | 5 | Dipicolinic Acid | 4 | PASS |
| 9 | Sodium Propionate | 50 | 46 | Diethyl ether | 0 | Dipicolinic Acid | 4 | PASS |
| 10 | Sodium Acetate | 25 | 36 | Diethyl ether | 10 | Dipicolinic Acid | 4 | PASS |
| | Sodium Formate | 25 | | | | | | |

### Example 15—Method for Preparing Peri-Peroxy Acid Sterilization Solutions from Alternative Carboxylic Acid Sources, a Reactive Oxygen Species, an MME, a Catalyst and a Stabilizer

Peri-peroxy acid solutions (35 g) were generated my mixing an alternative carboxylic acid source (Sodium Acetate, 99% (Sigma Aldrich, St. Louis, Mo.), Sodium Formate, 99% (Sigma Aldrich, St. Louis, Mo.) or Sodium Propionate (Sigma Aldrich, St. Louis, Mo.)), aqueous hydrogen peroxide, 30 wt % (Sigma Aldrich, St. Louis, Mo.) an MME (Diethyl Ether, 99.7% (Sigma Aldrich, St. Louis, Mo.)), a catalyst (Sulfuric Acid, 99.9999% (Sigma Aldrich, St. Louis, Mo.) and a stabilizer (Dipicolinic Acid, 99% (Sigma Aldrich, St. Louis, Mo.)) according to the embodiments described above. Reaction contents are shown in Table 8. The sterilant solutions are then transferred to a container or wrapping sufficiently porous to emit vapors.

### Example 16—Sterilization of a Surgical Scalpel, a Fabric, and a Foley Catheter Using Peri-Peroxy Acid Sterilant Solutions Generated from Alternative Carboxylic Acid Source, a Reactive Oxygen Species, an MME, a Catalyst and a Stabilizer

Peri-peroxy acid solutions (35 g) from Example 15, a scalpel, a piece of fabric, a foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to a base pressure of 200 mTorr while heat of 40° C. is evenly applied to the chamber. Once the base pressure is reached, the system is allowed to remain in a steady state of about 1 Torr for 15 minutes. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58°□C. after which time the color of the media is visualized for color change. Positive (PASS) results from a biological indicator test are seen when the color of the media does not change color. This result indicates the peri-peroxy acid sterilant solution under the descried sterilization chamber run conditions was able to successfully permeate through the porous transport filter of the biological indicator and achieve an SAL of 1×10E-6. Negative (FAIL) results from a biological indicator test are seen when the color of the media does change color, indicating an SAL of 1×10-6 was not achieved under the described sterilization process conditions. Results from biological indicator tests are listed in Table 8.

TABLE 8

| | | Alternative | Hydrogen | | | | | | | |
|---|---|---|---|---|---|---|---|---|---|---|
| | Alternative | Carboxylic | Peroxide | | | | | | | B.I. |
| | Carboxylic | Acid Source | (aq) | | MME | | Catalyst | | Stabilizer | Test |
| Formulation | Acid Source | (wt %) | (wt %) | MME | (wt %) | Catalyst | (wt %) | Stabilizer | (wt %) | Result |
| 1 | Sodium Acetate | 50 | 30.5 | Diethyl ether | 15 | Sulfuric Acid | 0.5 | Dipicolinic Acid | 4 | PASS |
| 2 | Sodium Acetate | 40 | 50.5 | Diethyl ether | 5 | Sulfuric Acid | 0.5 | Dipicolinic Acid | 4 | PASS |
| 3 | Sodium Acetate | 50 | 45.5 | Diethyl ether | 0 | Sulfuric Acid | 0.5 | Dipicolinic Acid | 4 | PASS |
| 4 | Sodium Formate | 50 | 30.5 | Diethyl ether | 15 | Sulfuric Acid | 0.5 | Dipicolinic Acid | 4 | PASS |
| 5 | Sodium Formate | 40 | 50.5 | Diethyl ether | 5 | Sulfuric Acid | 0.5 | Dipicolinic Acid | 4 | PASS |
| 6 | Sodium Formate | 50 | 45.5 | Diethyl ether | 0 | Sulfuric Acid | 0.5 | Dipicolinic Acid | 4 | PASS |
| 7 | Sodium Propionate | 50 | 30.5 | Diethyl ether | 15 | Sulfuric Acid | 0.5 | Dipicolinic Acid | 4 | PASS |
| 8 | Sodium Propionate | 40 | 50.5 | Diethyl ether | 5 | Sulfuric Acid | 0.5 | Dipicolinic Acid | 4 | PASS |
| 9 | Sodium Propionate | 50 | 45.5 | Diethyl ether | 0 | Sulfuric Acid | 0.5 | Dipicolinic Acid | 4 | PASS |
| 10 | Sodium Acetate | 25 | 35.5 | Diethyl ether | 10 | Sulfuric Acid | 0.5 | Dipicolinic Acid | 4 | PASS |
| | Sodium Formate | 25 | | | | | | | | |

### Examples of Peri-Peroxy Acid Sterilant Matrix Embodiments

Peri-peroxy acid sterilant matrices used in the examples listed below can be synthesized according to the described embodiments and illustrative examples described herein. The vapors of peri-peroxy acid are generated according to techniques, such as those described herein.

### Example 17—Method for Preparing Sterilization Gel from Peri-Peroxy Acid Sterilant Solutions and Polyethylene Glycols

Peri-peroxy acid sterilization gels (70 g) were generated my mixing a peri-peroxy acid with a polyethylene glycol (Polyethylene glycol Mn 400 (Sigma Aldrich, St. Louis, Mo.), Polyethylene glycol Mn 600 (Sigma Aldrich, St. Louis, Mo.), Polyethylene glycol Mn 1000 (Sigma Aldrich, St. Louis, Mo.) or Polyethylene glycol 3350 (Sigma Aldrich, St. Louis, Mo.)). Reaction contents are shown in Table 9. The sterilization gel is then transferred to a container or wrapping sufficiently porous to emit vapors. Peri-peroxy acid component materials, Acetic Acid, 98%, Formic Acid, 98%, Propionic Acid, 99.5%, aqueous Hydrogen Peroxide (30 wt %), Diethyl Ether, 99.7% and Methanol, 99.9% were purchased from Sigma Aldrich, St. Louis, Mo.

### Example 18—Sterilization of a Surgical Scalpel, Fabric, and a Foley Catheter Using Peri-Peroxy Acid Sterilization Gels

Sterilization gel (70 g) from Example 17, a scalpel, a piece of fabric, a foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to a base pressure of 200 mTorr while heat of 40° C. is evenly applied to the chamber. Once the base pressure is reached, the system is allowed to remain in a steady state of about 1 Torr for 15 minutes. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58°□C. after which time the color of the media is visualized for color change. Positive (PASS) results from a biological indicator test are seen when the color of the media does not change color. This result indicates the peri-peroxy acid sterilant solution under the descried sterilization chamber run conditions was able to successfully permeate through the porous transport filter of the biological indicator and achieve an SAL of 1×10E-6. Negative (FAIL) results from a biological indicator test are seen when the color of the media does change color, indicating an SAL of 1×10-6 was not achieved under the described sterilization process conditions. Results from biological indicator tests are listed in Table 9.

TABLE 9

| | Peri-peroxy | Peroxy acid | MME | Peri-peroxy acid | | Stabilization | B.I. Test |
|---|---|---|---|---|---|---|---|
| Formulation | acid Solution | (wt %) | (wt %) | Solution (wt %) | Stabilization Matrix | Matrix (wt %) | Result |
| 1 | Peracetic acid-Diethyl ether | 99 | 1 | 50 | Polyethylene glycol Mn 400 | 50 | PASS |
| 2 | Peracetic acid-Diethyl ether | 99 | 1 | 50 | Polyethylene glycol Mn 600 | 50 | PASS |
| 3 | Peracetic acid-Diethyl ether | 99 | 1 | 50 | Polyethylene glycol Mn 1000 | 50 | PASS |
| 4 | Peracetic acid-Diethyl ether | 99 | 1 | 50 | Polyethylene glycol Mn 3350 | 50 | PASS |
| 5 | Performic acid-Diethyl ether | 99 | 1 | 50 | Polyethylene glycol Mn 400 | 50 | PASS |
| 6 | Performic acid-Diethyl ether | 99 | 1 | 50 | Polyethylene glycol Mn 600 | 50 | PASS |
| 7 | Performic acid-Diethyl ether | 99 | 1 | 50 | Polyethylene glycol Mn 1000 | 50 | PASS |
| 8 | Performic acid-Diethyl ether | 99 | 1 | 50 | Polyethylene glycol Mn 3350 | 50 | PASS |
| 9 | Perpropionic acid-Diethyl ether | 99 | 1 | 50 | Polyethylene glycol Mn 400 | 50 | PASS |
| 10 | Perpropionic acid-Diethyl ether | 99 | 1 | 50 | Polyethylene glycol Mn 600 | 50 | PASS |
| 11 | Perpropionic acid-Diethyl ether | 99 | 1 | 50 | Polyethylene glycol Mn 1000 | 50 | PASS |
| 12 | Perpropionic acid-Diethyl ether | 99 | 1 | 50 | Polyethylene glycol Mn 3350 | 50 | PASS |
| 13 | Peracetic acid-Methanol | 99 | 1 | 50 | Polyethylene glycol Mn 400 | 50 | PASS |
| 14 | Peracetic acid-Methanol | 99 | 1 | 50 | Polyethylene glycol Mn 600 | 50 | PASS |
| 15 | Peracetic acid-Methanol | 99 | 1 | 50 | Polyethylene glycol Mn 1000 | 50 | PASS |
| 16 | Peracetic acid-Methanol | 99 | 1 | 50 | Polyethylene glycol Mn 3350 | 50 | PASS |
| 17 | Performic acid-Methanol | 99 | 1 | 50 | Polyethylene glycol Mn 400 | 50 | PASS |
| 18 | Performic acid-Methanol | 99 | 1 | 50 | Polyethylene glycol Mn 600 | 50 | PASS |
| 19 | Performic acid-Methanol | 99 | 1 | 50 | Polyethylene glycol Mn 1000 | 50 | PASS |
| 20 | Performic acid-Methanol | 99 | 1 | 50 | Polyethylene glycol Mn 3350 | 50 | PASS |
| 21 | Perpropionic acid-Methanol | 99 | 1 | 50 | Polyethylene glycol Mn 400 | 50 | PASS |
| 22 | Perpropionic acid-Methanol | 99 | 1 | 50 | Polyethylene glycol Mn 600 | 50 | PASS |
| 23 | Perpropionic acid-Methanol | 99 | 1 | 50 | Polyethylene glycol Mn 1000 | 50 | PASS |
| 24 | Perpropionic acid-Methanol | 99 | 1 | 50 | Polyethylene glycol Mn 3350 | 50 | PASS |

### Example 19—Method for Preparing Sterilization Solid from Peri-Peroxy Acid Sterilant Solution and Polyethylene Glycols

Peri-peroxy acid sterilization solids (70 g) were generated by melting together and mixing polyethylene glycol Mn 3350 (Sigma Aldrich, St. Louis, Mo.) and polyethylene glycol Mn 1000 (Sigma Aldrich, St. Louis, Mo.), as described in Table 10, by melting together both polyethylene glycols at 55° C. The mixtures were allowed to cool to room temperature after which time, the polyethylene glycol solid mixture was compounded with a peri-peroxy acid sterilant solution, as described in Table 10, to form a 70 g sterilization solid. The sterilization solid is then transferred to a container or wrapping sufficiently porous to emit vapors.

### Example 20—Sterilization of Surgical Scalpel, Fabric, and Foley Catheter Using Peri-Peroxy Acid Sterilization Solid

Sterilization solid (70 g) from Example 19, a scalpel, a piece of fabric, a foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to a base pressure of 200 mTorr while heat of 40° C. is evenly applied to the chamber. Once the base pressure is reached, the system is allowed to remain in a steady state of about 1 Torr for 15 minutes. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58°□C. after which time the color of the media is visualized for color change. Positive (PASS) results from a biological indicator test are seen when the color of the media does not change color. This result indicates the peri-peroxy acid sterilant solution under the descried sterilization chamber run conditions was able to successfully permeate through the porous transport filter of the biological indicator and achieve an SAL of 1×10E-6. Negative (FAIL) results from a biological indicator test are seen when the color of the media does change color, indicating an SAL of 1×10-6 was not achieved under the described sterilization process conditions. Results from biological indicator tests are listed in Table 10.

TABLE 10

| | | Peroxy acid | MME | Peri-peroxy acid | | Stablization | B.I. Test |
|---|---|---|---|---|---|---|---|
| Formulation | Peri-peroxy acid Solution | (wt %) | (wt %) | Solution (wt %) | Stabilization Matrix | Matrix (wt %) | Result |
| 1 | Peracetic acid-Diethyl ether | 99 | 1 | 25 | Polyethylene glycol Mn 3350- | 90 | PASS |
| | | | | | Polyethylene glycol Mn 1000 | 10 | |
| 2 | Peracetic acid-Diethyl ether | 99 | 1 | 15 | Polyethylene glycol Mn 3350- | 90 | PASS |
| | | | | | Polyethylene glycol Mn 1000 | 10 | |
| 3 | Peracetic acid-Diethyl ether | 99 | 1 | 25 | Polyethylene glycol Mn 3350- | 85 | PASS |
| | | | | | Polyethylene glycol Mn 1000 | 15 | |
| 4 | Peracetic aci -Diethyl ether | 99 | 1 | 15 | Polyethylene glycol Mn 3350- | 85 | PASS |
| | | | | | Polyethylene glycol Mn 1000 | 15 | |
| 5 | Performic acid-Diethyl ether | 99 | 1 | 25 | Polyethylene glycol Mn 3350- | 90 | PASS |
| | | | | | Polyethylene glycol Mn 1000 | 10 | |
| 6 | Performic acid-Diethyl ether | 99 | 1 | 15 | Polyethylene glycol Mn 3350- | 90 | PASS |
| | | | | | Polyethylene glycol Mn 1000 | 10 | |
| 7 | Performic acid-Diethyl ether | 99 | 1 | 25 | Polyethylene glycol Mn 3350- | 85 | PASS |
| | | | | | Polyethylene glycol Mn 1000 | 15 | |
| 8 | Performic acid-Diethyl ether | 99 | 1 | 15 | Polyethylene glycol Mn 3350- | 85 | PASS |
| | | | | | Polyethylene glycol Mn 1000 | 15 | |
| 9 | Perpropionic acid-Diethyl ether | 99 | 1 | 25 | Polyethylene glycol Mn 3350- | 90 | PASS |
| | | | | | Polyethylene glycol Mn 1000 | 10 | |
| 10 | Perpropionic acid-Diethyl ether | 99 | 1 | 15 | Polyethylene glycol Mn 3350- | 90 | PASS |
| | | | | | Polyethylene glycol Mn 1000 | 10 | |
| 11 | Perpropionic acid-Diethyl ether | 99 | 1 | 25 | Polyethylene glycol Mn 3350- | 85 | PASS |
| | | | | | Polyethylene glycol Mn 1000 | 15 | |
| 12 | Perpropionic acid-Diethyl ether | 99 | 1 | 15 | Polyethylene glycol Mn 3350- | 85 | PASS |
| | | | | | Polyethylene glycol Mn 1000 | 15 | |
| 13 | Peracetic acid-Methanol | 99 | 1 | 25 | Polyethylene glycol Mn 3350- | 90 | PASS |
| | | | | | Polyethylene glycol Mn 1000 | 10 | |
| 14 | Peracetic acid-Methanol | 99 | 1 | 15 | Polyethylene glycol Mn 3350- | 90 | PASS |
| | | | | | Polyethylene glycol Mn 1000 | 10 | |
| 15 | Peracetic acid-Methanol | 99 | 1 | 25 | Polyethylene glycol Mn 3350- | 85 | PASS |
| | | | | | Polyethylene glycol Mn 1000 | 15 | |
| 16 | Peracetic acid-Methanol | 99 | 1 | 15 | Polyethylene glycol Mn 3350- | 85 | PASS |
| | | | | | Polyethylene glycol Mn 1000 | 15 | |
| 17 | Performic acid-Methnol | 99 | 1 | 25 | Polyethylene glycol Mn 3350- | 90 | PASS |
| | | | | | Polyethylene glycol Mn 1000 | 10 | |
| 18 | Performic acid-Methanol | 99 | 1 | 15 | Polyethylene glycol Mn 3350- | 90 | PASS |
| | | | | | Polyethylene glycol Mn 1000 | 10 | |
| 19 | Performic acid-Methanol | 99 | 1 | 25 | Polyethylene glycol Mn 3350- | 85 | PASS |
| | | | | | Polyethylene glycol Mn 1000 | 15 | |
| 20 | Performic acid-Methanol | 99 | 1 | 15 | Polyethylene glycol Mn 3350- | 85 | PASS |
| | | | | | Polyethylene glycol Mn 1000 | 15 | |
| 21 | Perpropionic acid-Methanol | 99 | 1 | 25 | Polyethylene glycol Mn 3350- | 90 | PASS |
| | | | | | Polyethylene glycol Mn 1000 | 10 | |
| 22 | Perpropionic acid-Methanol | 99 | 1 | 15 | Polyethylene glycol Mn 3350- | 90 | PASS |
| | | | | | Polyethylene glycol Mn 1000 | 10 | |
| 23 | Perpropionic acid-Methanol | 99 | 1 | 25 | Polyethylene glycol Mn 3350- | 85 | PASS |
| | | | | | Polyethylene glycol Mn 1000 | 15 | |
| 24 | Perpropionic acid-Methanol | 99 | 1 | 15 | Polyethylene glycol Mn 3350- | 85 | PASS |
| | | | | | Polyethylene glycol Mn 1000 | 15 | |

### Examples of Package and Delivery Device Embodiments

### Example 21—Sterilization of a Surgical Scalpel, Fabric and Foley Catheter Using Peri-Peracetic Acid Sterilant Solution Generated from Ampoule (Type 1)

A scalpel, fabric, foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to 200 mTorr. The compartment containing 20 g acetic acid, 98% (Sigma Aldrich, St. Louis, Mo.) and the compartment containing 1.3 g diethyl ether, 99.7% (Sigma Aldrich, St. Louis, Mo.) within the ampoule housing containing 24 g aqueous hydrogen peroxide, 30 wt. % (Sigma Aldrich, St. Louis, Mo.) is broken releasing acetic acid and diethyl ether into the hydrogen peroxide forming the peri-peroxy acid sterilant solution. The ampoule housing containing the peri-peroxy acid sterilant solution is locked into the sterilizing system. Heat of 40° C. is evenly applied to the chamber. The preformed peri-peroxy acid is injected or dispensed into the system generating a peri-peroxy acid vapor within the system. The system is allowed to remain in a steady state of about 1 Torr for 15 minutes. At the end of the 15 minutes, the chamber is vented with argon gas until it reaches atmospheric pressure. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test were seen when the color of the media did not change color. This result indicates the peri-peroxy acid sterilant solution under the described sterilization chamber run conditions were able to successfully permeate through the porous transport filter of the biological indicator and achieve an SAL of 1×10E-6.

### Example 22—Sterilization of a Surgical Scalpel, Fabric and Foley Catheter Using Peri-Performic Acid Sterilant Solution Generated from Ampoule (Type 1)

A scalpel, fabric, foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to 200 mTorr. The compartment containing 20 g formic acid, 98% (Sigma Aldrich, St. Louis, Mo.) and the compartment containing 1.3 g diethyl ether, 99.7% (Sigma Aldrich, St. Louis, Mo.) within the ampoule housing containing 24 g aqueous hydrogen peroxide, 30 wt. % (Sigma Aldrich, St. Louis, Mo.) is broken releasing formic acid and diethyl ether into the hydrogen peroxide forming the peri-peroxy acid sterilant solution. The ampoule housing containing the peri-peroxy acid sterilant solution is locked into the sterilizing system. Heat of 40° C. is evenly applied to the chamber. The preformed peri-peroxy acid is injected or dispensed into the system generating a peri-peroxy acid vapor within the system. The system is allowed to remain in a steady state of about 1 Torr for 15 minutes. At the end of the 15 minutes, the chamber is vented with argon gas until it reaches atmospheric pressure. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test were seen when the color of the media did not change color. This result indicates the peri-peroxy acid sterilant solution under the described sterilization chamber run conditions were able to successfully permeate through the porous transport filter of the biological indicator and achieve an SAL of 1×10E-6.

### Example 23—Sterilization of a Surgical Scalpel, Fabric and Foley Catheter Using Peri-Propionic Acid Sterilant Solution Generated from Ampoule (Type 1)

A scalpel, fabric, foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to 200 mTorr. The compartment containing 20 g propionic acid, 99.5% (Sigma Aldrich, St. Louis, Mo.) and the compartment containing 1.3 g diethyl ether, 99.7% (Sigma Aldrich, St. Louis, Mo.) within the ampoule housing containing 24 g aqueous hydrogen peroxide, 30 wt. % (Sigma Aldrich, St. Louis, Mo.) is broken releasing propionic acid and diethyl ether into the hydrogen peroxide forming the peri-peroxy acid sterilant solution. The ampoule housing containing the peri-peroxy acid sterilant solution is locked into the sterilizing system. Heat of 40° C. is evenly applied to the chamber. The preformed peri-peroxy acid is injected or dispensed into the system generating a peri-peroxy acid vapor within the system. The system is allowed to remain in a steady state of about 1 Torr for 15 minutes. At the end of the 15 minutes, the chamber is vented with argon gas until it reaches atmospheric pressure. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test were seen when the color of the media did not change color. This result indicates the peri-peroxy acid sterilant solution under the described sterilization chamber run conditions were able to successfully permeate through the porous transport filter of the biological indicator and achieve an SAL of 1×10E-6.

### Example 24—Sterilization of a Surgical Scalpel, Fabric and Foley Catheter Using Peri-Peracetic Acid Sterilant Solution Generated from Ampoule (Type 2)

A scalpel, fabric, foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to 200 mTorr. The compartment containing 20 g acetic acid, 98% (Sigma Aldrich, St. Louis, Mo.), the compartment containing 1.3 g diethyl ether, 99.7% (Sigma Aldrich, St. Louis, Mo.), and the compartment containing 24 g aqueous hydrogen peroxide, 30 wt. % (Sigma Aldrich, St. Louis, Mo.) is broken releasing acetic acid, diethyl ether and hydrogen peroxide into a reservoir of the ampoule housing forming the peri-peroxy acid sterilant solution. The ampoule housing containing the peri-peroxy acid sterilant solution is locked into the sterilizing system. Heat of 40° C. is evenly applied to the chamber. The preformed peri-peroxy acid is injected or dispensed into the system generating a peri-peroxy acid vapor within the system. The system is allowed to remain in a steady state of about 1 Torr for 15 minutes. At the end of the 15 minutes, the chamber is vented with argon gas until it reaches atmospheric pressure. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test are seen when the color of the media did not change color. This result indicates the peri-peroxy acid sterilant solution under the described sterilization chamber run conditions are able to successfully permeate through the porous transport filter of the biological indicator and achieve an SAL of 1×10E-6.

### Example 25—Sterilization of a Surgical Scalpel, Fabric and Foley Catheter Using Peri-Performic Acid Sterilant Solution Generated from Ampoule (Type 2)

A scalpel, fabric, foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to 200 mTorr. The compartment containing 20 g formic acid, 98% (Sigma Aldrich, St. Louis, Mo.), the compartment containing 1.3 g diethyl ether, 99.7% (Sigma Aldrich, St. Louis, Mo.), and the compartment containing 24 g aqueous hydrogen peroxide, 30 wt. % (Sigma Aldrich, St. Louis, Mo.) is broken releasing formic acid, diethyl ether and hydrogen peroxide into a reservoir of the ampoule housing forming the peri-peroxy acid sterilant solution. The ampoule housing containing the peri-peroxy acid sterilant solution is locked into the sterilizing system. Heat of 40° C. is evenly applied to the chamber. The preformed peri-peroxy acid is injected or dispensed into the system generating a peri-peroxy acid vapor within the system. The system is allowed to remain in a steady state of about 1 Torr for 15 minutes. At the end of the 15 minutes, the chamber is vented with argon gas until it reaches atmospheric pressure. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test are seen when the color of the media did not change color. This result indicates the peri-peroxy acid sterilant solution under the described sterilization chamber run conditions are able to successfully permeate through the porous transport filter of the biological indicator and achieve an SAL of 1×10E-6.

### Example 26—Sterilization of a Surgical Scalpel, Fabric and Foley Catheter Using Peri-Perpropionic Acid Sterilant Solution Generated from Ampoule (Type 2)

A scalpel, fabric, foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to 200 mTorr. The compartment containing 20 g propionic acid, 98% (Sigma Aldrich, St. Louis, Mo.), the compartment containing 1.3 g diethyl ether, 99.7% (Sigma Aldrich, St. Louis, Mo.), and the compartment containing 24 g aqueous hydrogen peroxide, 30 wt. % (Sigma Aldrich, St. Louis, Mo.) is broken releasing propionic acid, diethyl ether and hydrogen peroxide into a reservoir of the ampoule housing forming the peri-peroxy acid sterilant solution. The ampoule housing containing the peri-peroxy acid sterilant solution is locked into the sterilizing system. Heat of 40° C. is evenly applied to the chamber. The preformed peri-peroxy acid is injected or dispensed into the system generating a peri-peroxy acid vapor within the system. The system is allowed to remain in a steady state of about 1 Torr for 15 minutes. At the end of the 15 minutes, the chamber is vented with argon gas until it reaches atmospheric pressure. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test are seen when the color of the media did not change color. This result indicates the peri-peroxy acid sterilant solution under the described sterilization chamber run conditions are able to successfully permeate through the porous transport filter of the biological indicator and achieve an SAL of 1×10E-6.

### Example 27—Sterilization of a Surgical Scalpel, Fabric and Foley Catheter Using Peri-Peracetic Acid Sterilant Solution Generated from Cartridges

A scalpel, fabric, foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. Three cartridges, one containing acetic acid, 98% (Sigma Aldrich, St. Louis, Mo.), one containing diethyl ether, 99.7% (Sigma Aldrich, St. Louis, Mo.) and one containing 30 wt % aqueous hydrogen peroxide (Sigma Aldrich, St. Louis, Mo.) are connected to the sterilization system. The chamber is then evacuated to 200 mTorr and heat of 40° C. is evenly applied to the sterilization system. After reaching base pressure, a 35 g mixture of peri-peracetic acid sterilant solution components containing a 45/10/45 wt % mixture of acetic acid/diethyl ether/hydrogen peroxide is injected into a chamber that is separate from the sterilization chamber. The peri-peroxy acid sterilant solution is allowed to form in the separate chamber after which point the peri-peroxy acid is vaporized under reduced pressure at 40° C. The pre-formed peri-peroxy acid sterilant vapor is injected or dispensed into the sterilization chamber. The system is allowed to remain in a steady state of about 1 Torr for 15 minutes. At the end of the 15 minutes, the chamber is vented with argon gas until it reaches atmospheric pressure. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test are seen when the color of the media did not change color. This result indicates the peri-peroxy acid sterilant solution under the described sterilization chamber run conditions are able to successfully permeate through the porous transport filter of the biological indicator and achieve an SAL of 1×10E-6.

### Example 28—Sterilization of a Surgical Scalpel, Fabric and Foley Catheter Using Peri-Performic Acid Sterilant Solution Generated from Cartridges

A scalpel, fabric, foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. Three cartridges, one containing formic acid, 98% (Sigma Aldrich, St. Louis, Mo.), one containing diethyl ether, 99.7% (Sigma Aldrich, St. Louis, Mo.) and one containing 30 wt % aqueous hydrogen peroxide (Sigma Aldrich, St. Louis, Mo.) are connected to the sterilization system. The chamber is then evacuated to 200 mTorr and heat of 40° C. is evenly applied to the sterilization system. After reaching base pressure, a 35 g mixture of peri-performic acid sterilant solution components containing a 45/10/45 wt % mixture of formic acid/diethyl ether/hydrogen peroxide is injected into a chamber that is separate from the sterilization chamber. The peri-peroxy acid sterilant solution is allowed to form in the separate chamber after which point the peri-peroxy acid is vaporized under reduced pressure at 40° C. The pre-formed peri-peroxy acid sterilant vapor is injected or dispensed into the sterilization chamber. The system is allowed to remain in a steady state of about 1 Torr for 15 minutes. At the end of the 15 minutes, the chamber is vented with argon gas until it reaches atmospheric pressure. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test are seen when the color of the media did not change color. This result indicates the peri-peroxy acid sterilant solution under the described sterilization chamber run conditions are able to successfully permeate through the porous transport filter of the biological indicator and achieve an SAL of 1×10E-6.

### Example 29—Sterilization of a Surgical Scalpel, Fabric and Foley Catheter Using Peri-Perpropionic Acid Sterilant Solution Generated from Cartridges

A scalpel, fabric, foley catheter and self-contained biological indicator ampoules (MesaLabs, Bozeman, Mont.) containing a stainless steel disc inoculated with a Geobacillus stearothermophilus spore SAL population of 1×10E6 are placed in the sterilization chamber. Three cartridges, one containing propionic acid, 98% (Sigma Aldrich, St. Louis, Mo.), one containing diethyl ether, 99.7% (Sigma Aldrich, St. Louis, Mo.) and one containing 30 wt % aqueous hydrogen peroxide (Sigma Aldrich, St. Louis, Mo.) are connected to the sterilization system. The chamber is then evacuated to 200 mTorr and heat of 40° C. is evenly applied to the sterilization system. After reaching base pressure, a 35 g mixture of peri-propionic acid sterilant solution components containing a 45/10/45 wt % mixture of propionic acid/diethyl ether/hydrogen peroxide is injected into a chamber that is separate from the sterilization chamber. The peri-peroxy acid sterilant solution is allowed to form in the separate chamber after which point the peri-peroxy acid is vaporized under reduced pressure at 40° C. The pre-formed peri-peroxy acid sterilant vapor is injected or dispensed into the sterilization chamber. The system is allowed to remain in a steady state of about 1 Torr for 15 minutes. At the end of the 15 minutes, the chamber is vented with argon gas until it reaches atmospheric pressure. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test are seen when the color of the media did not change color. This result indicates the peri-peroxy acid sterilant solution under the described sterilization chamber run conditions are able to successfully permeate through the porous transport filter of the biological indicator and achieve an SAL of 1×10E-6.

### Example 30—Method of Preparing and Analyzing the Peroxy Acid Component of Peri-Peroxy Acid Sterilant Solutions

The peri-peracetic acid sterilant solution component peracetic acid can be synthesized by mixing 6.6 g of aqueous hydrogen peroxide, 30 wt % (Sigma Aldrich, St. Louis, Mo.) with 4.6 g acetic acid acid, 98% (Sigma Aldrich, St. Louis, Mo.) followed by the addition of 1.2 g sulfuric acid, 99.9999% (Sigma Aldrich, St. Louis, Mo.). The peracetic acid sterilant solution was allowed to come to equilibrium at room temperature.

The peri-performic acid sterilant solution component performic acid can be synthesized by mixing 6.6 g aqueous hydrogen peroxide, 30 wt % (Sigma Aldrich, St. Louis, Mo.) with 5.4 g formic acid, 98% (Sigma Aldrich, St. Louis, Mo.) followed by the addition of 1.2 g sulfuric acid, 99.9999% (Sigma Aldrich, St. Louis, Mo.). The performic acid sterilant solution was allowed to come to equilibrium at room temperature.

The peri-propionic acid sterilant solution component perpropionic acid can be synthesized by mixing 6.6 g aqueous hydrogen peroxide, 30 wt % (Sigma Aldrich, St. Louis, Mo.) with 4.4 g propionic acid, 98% (Sigma Aldrich, St. Louis, Mo.) followed by the addition of 1.2 g sulfuric acid, 99.9999% (Sigma Aldrich, St. Louis, Mo.). The propionic acid sterilant solution was allowed to come to equilibrium at room temperature.

Peri-peroxy acid solutions were generated my mixing a carboxylic acid (Acetic Acid, 98% (Sigma Aldrich, St. Louis, Mo.) or Formic Acid, 98% (Sigma Aldrich, St. Louis, Mo.), 50 wt % aqueous hydrogen peroxide (Sigma Aldrich, St. Louis, Mo.), sulfuric acid, 99.9999% (Sigma Aldrich, St. Louis, Mo.) and an MME (Diethyl Ether, 99.7% (Sigma Aldrich, St. Louis, Mo.) or Methanol, 99.9% (Sigma Aldrich, St. Louis, Mo.)) according to the embodiments described above. Peroxy acid solutions were generated by mixing a carboxylic acid (Acetic Acid, 98% (Sigma Aldrich, St. Louis, Mo.) or Formic Acid, 98% (Sigma Aldrich, St. Louis, Mo.), 50 wt % aqueous hydrogen peroxide (Sigma Aldrich, St. Louis, Mo.), sulfuric acid, 99.9999% (Sigma Aldrich, St. Louis, Mo.) according to the embodiments described herein. Reaction contents are shown in Table 11.

The total performic acid content and total hydrogen peroxide content of the sterilant solution was performed using a high-throughput microtiter plate based cerium sulfate and iodine method performed by Putt, K et al. in PLOS One, November 2013, Vol. 8, Issue 11.

TABLE 11

| | Carboxylic Acid | Hydrogen Peroxide | | Catalyst | | MME | Peroxy acid |
|---|---|---|---|---|---|---|---|
| Carboxylic Acid | Mass (g) | Mass (g) | Catalyst | Mass (g) | MME | Mass (g) | % Purity |
| Acetic Acid | 0.3514 | 0.2586 | Sulfuric Acid | 0.0098 | Diethyl Ether | 0 | 9.92 |
| Acetic Acid | 0.3514 | 0.2586 | Sulfuric Acid | 0.0098 | Diethyl Ether | 0.0220 | 9.61 |
| Formic Acid | 0.2696 | 0.2586 | Sulfuric Acid | 0.0098 | Methanol | 0 | 9.40 |
| Formic Acid | 0.2696 | 0.2586 | Sulfuric Acid | 0.0098 | Methanol | 0.003722 | 10.98 |

### Examples of Sterilant Matrix Embodiments

Performic acid used in the examples listed below can be synthesized by mixing 17.5 g aqueous hydrogen peroxide, 30 wt % (Sigma Aldrich, St. Louis, Mo.) with 17.5 g formic Acid, 90% (Sigma Aldrich, St. Louis, Mo.). The vapors of performic acid are generated according to techniques, such as those described herein.

### Example 31—Method for Preparing Sterilization Gel from Performic Acid and Polyethylene Glycol Mn 1000

70 g of a 50/50 (w/w) polyethylene glycol 1000 (Sigma Aldrich, St. Louis, Mo.) and performic acid is created. This mixture is stirred at 300 RPM on a stir plate at room temperature for 30 minutes. The gel is then transferred to a container or wrapping sufficiently porous to emit vapors.

### Example 32—Sterilization of a Surgical Scalpel Using Performic Acid Sterilization Gel

Performic acid sterilization gel (70 g) from Example 31, a scalpel and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to a base pressure of 200 mTorr while heat of 40° C. is evenly applied to the chamber. Once the base pressure is reached, the system is allowed to remain in a steady state of about 1 Torr for 15 minutes. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test are seen when the color of the media does not change color. This result indicates the performic acid sterilant solution under the descried sterilization chamber run conditions was able to successfully permeate through the porous transport filter of the biological indicator and achieve an SAL of 1×10E-6. Negative (FAIL) results from a biological indicator test are seem when the color of the media does change color, indicating an SAL of 1×10-6 was not achieved under the described sterilization process conditions. Results from biological indicator tests reveal sterilization at an SAL of 1×10-6.

### Example 33—Method for Preparing Sterilization Solid from Performic Acid and Polyethylene Glycol Mn 1000 and Polyethylene Glycol Mn 3350

A 105 g mixture of 85/15 w/w polyethylene glycol 3350 (Sigma Aldrich, St. Louis, Mo.) and polyethylene glycol 1000 (Sigma Aldrich, St. Louis, Mo.) is created by melting both polyethylene glycols in a glass beaker at 58° C. while stirring at 300 RPM on a heating stir plate for 30 minutes. After time let mixture cool to room temperature. The 85/15 w/w polyethylene glycol solid is compounded with 35 g of performic acid to form a solid.

### Example 34—Sterilization of Surgical Scalpel Using Performic Acid Sterilization Solid

Performic acid sterilization solid (70 g) from Example 33, a scalpel and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to a base pressure of 200 mTorr while heat of 40° C. is evenly applied to the chamber. Once the base pressure is reached, the system is allowed to remain in a steady state of about 1 Torr for 15 minutes. At the end of the process run, the chamber is brought to atmosphere and the scalpel, fabric, catheter and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test are seen when the color of the media does not change color. This result indicates the performic acid sterilant solution under the descried sterilization chamber run conditions was able to successfully permeate through the porous transport filter of the biological indicator and achieve an SAL of 1×10E-6. Negative (FAIL) results from a biological indicator test are seem when the color of the media does change color, indicating an SAL of 1×10-6 was not achieved under the described sterilization process conditions. Results from biological indicator tests reveal sterilization at an SAL of 1×10-6.

### Example 35—Sterilization of a Surgical Scalpel Using Performic Acid Generated from Ampoule (Type 1)

A scalpel and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to 200 mTorr. The compartment containing 20 g formic acid, 98% (Sigma Aldrich, St. Louis, Mo.) within the ampoule housing containing 24 g aqueous hydrogen peroxide, 30 wt. % (Sigma Aldrich, St. Louis, Mo.) is broken releasing formic acid into the hydrogen peroxide forming the performic acid sterilant solution. The ampoule housing containing the performic acid sterilant solution is locked into the sterilizing system. Heat of 40° C. is evenly applied to the chamber. The preformed performic acid is injected or dispensed into the system generating a performic acid vapor within the system. The system is allowed to remain in a steady state of about 1 Torr for 15 minutes. At the end of the 15 minutes, the chamber is vented with argon gas until it reaches atmospheric pressure. At the end of the process run, the chamber is brought to atmosphere and the scalpel and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test were seen when the color of the media did not change color. This result indicates the performic acid sterilant solution under the described sterilization chamber run conditions were able to successfully permeate through the porous transport filter of the biological indicator and achieve an SAL of 1×10E-6.

### Example 36—Sterilization of a Surgical Scalpel Using Performic Acid Generated from Ampoule (Type 2)

A scalpel and self-contained biological indicator ampoules (MesaLabs, Bozeman, Mont.) containing a stainless steel disc inoculated with a Geobacillus stearothermophilus spore SAL population of 1×10E6 are placed in the sterilization chamber. The chamber is then evacuated to 200 mTorr. The compartment containing 20 g formic acid, 98% (Sigma Aldrich, St. Louis, Mo.) and the compartment containing 24 g aqueous hydrogen peroxide, 30 wt. % (Sigma Aldrich, St. Louis, Mo.) is broken releasing formic acid and hydrogen peroxide into a reservoir of the ampoule housing forming the performic acid acid sterilant solution. The ampoule housing containing the performic acid acid sterilant solution is locked into the sterilizing system. Heat of 40° C. is evenly applied to the chamber. The preformed performic acid is injected or dispensed into the system generating a performic acid vapor within the system. The system is allowed to remain in a steady state of about 1 Torr for 15 minutes. At the end of the 15 minutes, the chamber is vented with argon gas until it reaches atmospheric pressure. At the end of the process run, the chamber is brought to atmosphere and the scalpel and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test are seen when the color of the media did not change color. This result indicates the performic acid sterilant solution under the described sterilization chamber run conditions are able to successfully permeate through the porous transport filter of the biological indicator and achieve an SAL of 1×10E-6.

### Example 37—Sterilization of a Surgical Scalpel Using Performic Acid Sterilant Solution Generated from Cartridges

A scalpel and self-contained biological indicator ampoules (MesaLabs, Bozeman, Mont.) containing a stainless steel disc inoculated with a Geobacillus stearothermophilus spore SAL population of 1×10E6 are placed in the sterilization chamber. Two cartridges, one containing formic acid, 98% (Sigma Aldrich, St. Louis, Mo.) and one containing 30 wt % aqueous hydrogen peroxide (Sigma Aldrich, St. Louis, Mo.) are connected to the sterilization system. The chamber is then evacuated to 200 mTorr and heat of 40° C. is evenly applied to the sterilization system. After reaching base pressure, a 35 g mixture of performic acid sterilant solution components containing a 45/55 wt % mixture of formic acid/hydrogen peroxide is injected into a chamber that is separate from the sterilization chamber. The performic acid sterilant solution is allowed to form in the separate chamber after which point the performic acid is vaporized under reduced pressure at 40° C. The pre-formed performic acid sterilant vapor is injected or dispensed into the sterilization chamber. The system is allowed to remain in a steady state of about 1 Torr for 15 minutes. At the end of the 15 minutes, the chamber is vented with argon gas until it reaches atmospheric pressure. At the end of the process run, the chamber is brought to atmosphere and the scalpel and biological indicators are removed from the chamber using an established sterile technique. The stainless steel disc inside each of the biological indicators are tested for SAL within the biological indicator by gently crushing the self-contained sealed-glass ampoule containing soybean casein digest culture media with color indicator to fully immerse the stainless steel disc and then incubating the biological indicator for 24 hours at 58° C. after which time the color of the media is visualized for color change. Positive (PASS) results from a biological indicator test are seen when the color of the media did not change color. This result indicates the performic acid sterilant solution under the described sterilization chamber run conditions are able to successfully permeate through the porous transport filter of the biological indicator and achieve an SAL of 1×10E-6.

The scalpel and a self-contained biological indicator ampoule (MesaLabs, Bozeman, Mont.) containing a SAL strip with a spore Geobacillus stearothermophilus population of 106 are placed in the sterilization chamber as described above. The cartridges containing formic acid and hydrogen peroxide are locked into the sterilizing system. The chamber is then evacuated to 1×10⁻³ Torr. Once base pressure is reached, a 50/50 mixture of performic acid is generated by injecting and mixing equal volumes of formic acid, 3 85% (Sigma Aldrich, St. Louis, Mo.) and hydrogen peroxide, 30 wt ¾ (Sigma Aldrich, St. Louis, Mo.) from their respective cartridges.

Heat of 40° C. is evenly applied to the chamber. The system is allowed to remain in a steady state of about 1 Torr for 15 minutes. At the end of the 15 minutes, the chamber is vented with argon gas until it reaches atmospheric pressure. At the end of the 15 minutes, the chamber is vented with argon gas until it reaches atmospheric pressure. The scalpel and biological indicator ampoule are removed using well-established sterile techniques. The SAL strip is tested within the biological indicator by gently crushing the ampoule and then incubating the biological indicator ampoule for 24 hours at 60° C. Following time, it will be found to be acceptably sterile at the 10-6 level.

Other examples and implementations are within the scope and spirit of the disclosure and appended claims. For example, features implementing functions can also be physically located at various positions, including being distributed such that portions of functions are implemented at different physical locations. Also, as used herein, including in the claims, “or” as used in a list of items prefaced by “at least one of” indicates a disjunctive list such that, for example, a list of “at least one of A, B, or C” means A or B or C or AB or AC or BC or ABC (i.e., A and B and C). Further, the term “exemplary” does not mean that the described example is preferred or better than other examples.

Various changes, substitutions, and alterations to the techniques described herein can be made without departing from the technology of the teachings as defined by the appended claims. Moreover, the scope of the disclosure and claims is not limited to the particular aspects of the process, machine, manufacture, composition of matter, means, methods, and actions described above. Processes, machines, manufacture, compositions of matter, means, methods, or actions, presently existing or later to be developed, that perform substantially the same function or achieve substantially the same result as the corresponding aspects described herein can be utilized. Accordingly, the appended claims include within their scope such processes, machines, manufacture, compositions of matter, means, methods, or actions.

1.-85. (canceled)

86. A method for providing a sterilant vapor to an enclosure for sterilizing or sanitizing the enclosure and/or an article provided within the enclosure, the method comprising:
exposing a mixture comprising a molecular mobility enhancer (MME) and a sterilant to the enclosure at a sub-atmospheric pressure condition, thereby providing the sterilant, the MME or both as a vapor in the enclosure.

87. The method of any one of claim 86, wherein the sterilant, the MME or both are-a liquid or a solid at normal temperature and pressure (NPT, 20° C. and 760 torr).

88. The method of claim 86, further comprising heating the mixture during the exposing step.

89. The method of claim 86, further comprising providing a carrier gas flow in fluid communication with the mixture and flowing the carrier gas flow in fluid communication with the mixture into the enclosure.

90. The method of claim 86, further comprising maintaining the enclosure at a pressure selected over the range of 0.1 torr to 200 torr for a time period selected from the range of 1 minute to 24 hours.

91. The method of claim 86, wherein the exposing step comprises providing the mixture in the enclosure followed by decreasing the pressure of the enclosure to below 760 torr.

92. The method of claim 86 for sterilizing the article, wherein the article is contacted with a vapor comprising the sterilant and the MME under vacuum at a selected pressure of the vapor for a selected time.

93. A solid-form sterilant, comprising
a sterilant matrix;
a sterilant compound encapsulated within the sterilant matrix; and
a molecular mobility enhancing (MME) agent encapsulated within the sterilant matrix.

94. The solid-form sterilant of claim 93, wherein the sterilant compound is selected from the group consisting of an acid, an alcohol, hydrogen peroxide, glutaraldehyde, ortho-phthaladehyde, and combinations thereof.

95. The solid-form sterilant of claim 94, wherein the sterilant compound is selected from the group consisting of a peri-peroxy acid, a phenolic acid, hypochlorous acid, isopropanol, hydrogen peroxide, glutaraldehyde, ortho-phthaladehyde, and combinations thereof.

96. The solid-form sterilant of claim 93, wherein the sterilant compound is a peri-peroxic acid selected from the group consisting of performic acid, a peroxy acid, and combinations thereof.

97. The solid-form sterilant of claim 96, where the peroxy acid is selected from the group consisting of saturated and unsaturated peroxy acids having between 1 and 8 carbon atoms and including any halogenated forms of the peroxy acids.

98. The solid-form sterilant of claim 96, where the peroxy acid is selected from the group consisting of peroxyacetic acid and its halogenated derivatives, peroxypropionic acid, and its halogenated derivatives and peroxybutyric acid and its halogenated derivatives.

99. The solid-form sterilant of claim 96, wherein the MME agent is selected from the group consisting of ether, alcohols, phenols, esters and amides.

100. The solid-form sterilant of claim 96, wherein the sterilant matrix comprises a matrix material selected from the group consisting of a polymer, a ceramic, a glass, and combinations thereof.

101. A packaged sterilant for use in a vacuum environment, comprising
a sterilant compound disposed within a package; and
a molecular mobility enhancing (MME) agent encapsulated within the package.

102. A method for providing a sterilant to an enclosure, the method comprising:
exposing a mixture comprising a molecular mobility enhancer (MME) and the sterilant to the enclosure at a sub-atmospheric pressure condition, thereby providing the sterilant as a vapor in the enclosure, wherein the mixture is a solid-form sterilant of claim 96.

103. The method of claim 102, wherein the enclosure is within an apparatus capable of achieving reduced pressure, and wherein the method comprises:
introducing a mixture of the sterilant and the molecular mobility enhancer into the apparatus, and
reducing the pressure in at least a portion of the apparatus to generate a vapor comprising sterilant in at least a portion of the apparatus.

104. The method of claim 103, wherein an article to be treated is present in the apparatus and the vapor generated is in contact with the article.

105. The method of claim 104, wherein the article to be treated comprises diffusion resistant surfaces.
